(19) United States
(12) Patent Application Publication
(10) Pub. No.: US 2002/0015697 A1
(43) Pub. Date: Feb. 7, 2002

(54) Biocidal methods and compositions

(76) Inventors: Kenneth Beckman, Alameda, CA (US); John L. Grimsich, Berkeley, CA (US); Christopher Hawk, Pittsburg, CA (US); Frank Swenson, Alameda, CA (US); David Tyler, Alameda, CA (US)

Correspondence Address: LAW OFFICES OF JONATHAN ALAN QUINE, P O BOX 458, ALAMEDA, CA, 94501

(21) Appl. No.: 09/804,694

(22) Filed: Mar. 14, 2001

Related U.S. Application Data
(60) Provisional application No. 60/188,981, filed on Mar. 13, 2000.
(60) Provisional application No. 60/188,995, filed on Mar. 13, 2000.
(60) Provisional application No. 60/188,783, filed on Mar. 13, 2000.
(60) Provisional application No. 60/220,618, filed on Jul. 25, 2000.

(51) Int. Cl. A61K 38/44; A61K 33/26; A61K 31/715; A61L 9/00

(52) U.S. Cl. 424/94.4; 514/54; 424/646; 422/30

(57) ABSTRACT

Methods, compositions, and kits for reducing a microbial population on a surface are provided. The microbial populations which can be treated using the methods, compositions, and kits of the present invention include prokaryotic, viral, and protozoan populations. The methods, compositions, and kits of the present invention have a number of uses in the fields of food production and medicine.

### CROSS-REFERENCES TO RELATED APPLICATIONS

This application is related to U.S. provisional patent applications 60/188,981 (filed Mar. 13, 2000); 60/188,995 (filed Mar. 13, 2000); 60/188,783 (filed Mar. 13, 2000) and 60/220,618 (filed Jul. 25, 2000). The present application claims priority to, and benefit of, these applications, pursuant to 35 U.S.C. §119(e) and any other applicable statute or rule.

### BACKGROUND OF THE INVENTION

Microbial infections represent a ubiquitous, constant, and grave threat to human health. As an example, according to the U.S. Centers for Disease Control (CDC), food poisoning due to enteric infections (many of which are of bacterial origin) has been estimated to cause 9,000 deaths annually in the United States, with between 6 and 80 million cases per year total. Foodborne infections may be caused by microbes originating from the food itself (e.g., microbial organisms derived from soil and fecal matter, bacteria released from the intestines of animals during processing) or from contamination introduced during the processing and preparation of food through its contact with contaminated surfaces, equipment or workers. Chief among the bacterial genera responsible for outbreaks of food poisoning are Escherichia, Salmonella, Shigella, Campylobacter, and Listeria. Systemic infections, introduced, for example, through wounds and burns, are also major killers. In 1997, septicemia (systemic bacterial infection) was the twelfth leading cause of death in the United States, according to the CDC, causing roughly 22,500 deaths. Species of Staphylococcus, Escherichia, Streptomyces and other bacteria are involved in systemic infections.

Of central importance in the fight against bacterial infections are preventative steps taken to eliminate viable pathogenic bacteria on foods, and to destroy bacteria in wounds and burns. Antibiotics have commonly been used as a proactive treatment, as well as post-infection. However, the rise in antibiotic-resistant strains of numerous bacterial species, such as vancomycin-resistant forms of Enterococci and methicillin-resistant Staphylococci, are becoming more common. The acquisition of antibiotic resistance means that common infections, which were once a nuisance but ultimately treatable, now represent potentially lethal events. In fact, hospitals themselves have become central to the spread of the worst strains of antibiotic-resistant microbes (so-called nosocomial infections), since individuals afflicted with these strains are often confined to hospital beds, and thereby represent a pool of antibiotic resistant microbes ready to infect patients who come into hospitals with fresh wounds or for surgery. As such, novel disinfectants are needed that attack a broad spectrum of microbial species (as antiseptics do) while defying attempts by the species to evolve resistance. The methods, compositions, and kits of the present invention address this need by providing novel mechanisms for the reduction of microbial populations and disinfection of surfaces.

### SUMMARY OF THE INVENTION

The present invention provides methods for reducing a microbial population on a surface. The methods include the steps of a) providing a low concentration of free iron; b) providing a non-oxidant stress inducer; and c) bringing the surface into contact with the low concentration of free iron and the non-oxidant stress inducer, thereby reducing the microbial population on the surface.

Optionally, the free iron used in the methods of the present invention can be either ferrous iron or ferric iron. A range of concentrations of free iron can be employed in the methods. For example, the low concentration of free iron can range between about 0.1 μM and about 100 mM free iron, preferably between about 0.1 μM and about 1 mM free iron. More preferably, the concentration of free iron used in the method is approximately 1μM free iron.

In one embodiment of the methods of the present invention, the free iron and the non-oxidant stress inducer are coadministered to the surface to be treated. In an alternative embodiment, a portion of the free iron is removed prior to administration of the non-oxidant stress inducer. Approximately 50%, 75%, 90%, 95%, 98%, 99%, 99.5%, or substantially all of the low concentration of free iron is removed prior to bringing the surface into contact with the non-oxidant stress inducer.

A number of non-oxidizing stress inducers can be employed in the method of the present invention. In one embodiment, the non-oxidizing stress inducer comprises one or more enzymes, for example, lysozyme. In another embodiment, the non-oxidizing stress inducer includes one or more polysaccharides, such as chitin, chitosan, polymannans (such as β(1−>4) acetyl mannan). Alternatively, the non-oxidizing stress inducers include, but are not limited to, various hypo-osmotic solutions, hyper-osmotic solutions, changes in pressure, changes in pH, and the like.

The methods of the present invention can further include the step of providing one or more biocide enhancers. Exemplary biocide enhancers include, but are not limited to, riboflavin, flavenoids, photo-activatable compounds, phenols, cetyl pyridinium chloride, trisodium phosphate, hydrogen peroxide, bleach, one or more fatty acids, one or more organic acids, citric acid, and ascorbic acid.

The microbial population can be reduced on a variety of surfaces using the methods of the present invention. For example, the surface can include a food surface, such as nuts, fruits or vegetables. The food surface can be treated pre-harvest, or it can be treated post-harvest. Alternatively the surface can comprises an animal surface, such as an exterior, or outer body surface, as well as an interior body surface (such as the digestive tract, lungs, or organ surfaces). Optionally, combinations of exterior and interior surfaces can be treated using the methods of the present invention. Further surfaces which can be treated to reduce the microbial population include, but are not limited to a skin surface (e.g., an epidermal surface, a mucosal surface, a wound, an abrasion, a burn, or a damaged region of tissue), an environmental surface (e.g., a countertop, a public restroom), a piece of medical equipment, and the like.

The methods of the present invention can further include the step of providing a siderophore. Exemplary siderophores include, but are not limited to, aerobactin, alcaligin, cepabactin, desferriferrichrysin, desferriferricrocin, desferriferrioxamine B, desferriferrioxamine E, coprogen, corrugatin, enterobactin, enterochelin, exochelin, ferrichrome, ferrioxamine, gallichrome, mycobaction, myxochelin, nocardamine, pseudobactin M114, pyoverdine, pyochelin, pseudobactin St3, rhizoferrin, rhodotorulic acid, schizokinen, pseudobactin 7NSK2, trencam, WCS, and vibriobactin.

The present invention also provides methods for reducing a microbial population on a food surface or a living tissue, by a) providing a composition comprising free iron; b) providing a non-oxidant stress inducer; and c) bringing the food surface or the living tissue into contact with the free iron and the non-oxidant stress inducer, thereby reducing the microbial population on the food surface or living tissue. A great range of concentrations can be used in these embodiments of the present invention, ranging from 0.1 μM to 1M free iron. The free iron and the non-oxidant stress inducer can be coadministered to the food surface or living tissue, or they may be sequentially administered. Optionally, a portion of the free iron (approximately 50%, 75%, 90%, 95%, 98%, 99%, 99.5%, or substantially all) is removed prior to administration of the non-oxidant stress inducer. The food surface or living tissue is exposed to the free iron composition and/or the non-oxidizing stress inducer for a variable length of time, ranging from a few minutes (e.g., rinsing a piece of fruit) to several hours to days (e.g. treating a wound or incision).

In one embodiment of the methods of the present invention, the step of providing the non-oxidizing stress inducer comprises exposing the food surface or living tissue to, for example, heat, irradiation, or osmotic shock. Optionally, the methods further include providing one or more siderophores, acids, bases, disinfectants, halides, organic solvents, oxidants, enzymes, antimicrobial agents, antibiotics, antiseptics, denaturants, or other biocide enhancers. Additional biocide enhancers include, but are not limited to, riboflavin, flavenoids, photo-activatable compounds, cetyl pyridinium chloride, trisodium phosphate, hydrogen peroxide, bleach, one or more fatty acids, organic acids, citric acid, and ascorbic acid.

Furthermore, the present invention provides methods for reducing a microbial population on a surface, including the steps of a) providing a preparation comprising free iron; b) providing one or more siderophores; and c) bringing the surface into contact with the preparation, thereby reducing the microbial population on the surface. Optionally, a range of concentrations of free iron (for example, between about 0.1 nM and about 1 M of either ferrous iron or ferric iron) can be employed in the methods. siderophores which can be employed in the methods of the present invention include, but are not limited to, aerobactin, enterobactin, and the like. The methods can be used to reduce the microbial population on a variety of surfaces, such as a food surface, an animal surface (e.g., an outer body surface, a digestive tract, or a combination thereof), an environmental surface, a piece of medical equipment, a wound, an abrasion, a burn, or a damaged region of tissue.

The present invention also provides novel free iron compositions for reducing a microbial population on a surface. Antimicrobial compositions including free iron and a siderophore are provided by the present invention, as are antimicrobial compositions including free iron and a polysaccharide, such as chitin or chitosan. In one embodiment, the composition of the present invention includes a preparation of free iron and a wound dressing component. In an alternative embodiment of the present invention, the composition includes a preparation of free iron and a lubricant or lotion. In a further embodiment of the present invention, the composition includes a preparation of free iron and an sexually-transmitted disease (STD) treatment component. In yet another embodiment of the present invention, the composition of the present invention includes a preparation of free iron and an oral rinse. The compositions of the present invention can further include one or more non-oxidant stress inducers, one or more iron-binding compounds (such as chelators and/or siderophores), or combinations thereof.

### BRIEF DESCRIPTION ON THE DRAWINGS

FIG. 1A is a graph depicting absorbance readings at 600 nm over time for a number of starting concentrations of E. coli cell cultures; FIG. 1B is the same data, plotted as function of initial cell density.

FIG. 2, panels A and B, are graphs showing the effects of free iron and hydrogen peroxide on cell growth, as measured by absorbance at 600 nm.

FIG. 3 depicts the effects of free iron and hydrogen peroxide prepared in an unbuffered, hypo-osmotic solution on cell growth.

FIG. 4 depicts effects of free iron prepared in an unbuffered, hypo-osmotic solution, and in the absence of a stress inducer, on cell growth

FIG. 5 depicts the effects of free iron and cetyl pyridinium chloride on cell growth.

FIG. 6 depicts the effects of free iron and citric acid on cell growth.

FIG. 7 depicts the effects of free iron and hypochlorite on cell growth.

FIG. 8 depicts the effects of free iron and lysozyme on cell growth.

FIG. 9 depicts the effects of free iron and TSP on cell growth.

### DETAILED DISCUSSION

Definitions

Before describing the present invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, reference to “a siderophore” includes mixtures of siderophores, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

As used herein, the term “siderophore” refers to transition metal-binding factors utilized (and often produced) by microbes (both prokaryotic and eukaryotic) for sequestering the transition metal (for example, iron) from the environment, which can then be actively transported into the cell by siderophore-binding cell surface receptors. Chelating compounds which do not undergo active transport into the cell are not considered siderophores for purposes of the present invention.

The term “microbe” refers to any unicellular organism or multicellular parasitic organism that one of skill in the art chooses to reduce in population.

The term “antimicrobial” as used herein refers to a compound, treatment, or effect that is biocidal (e.g., kills cells or components of cells), biostatic (e.g., prevents further growth of cells), or a combination thereof. As such, a classification as an “antimicrobial compounds” is meant to encompass, but is not limited to, compounds having bacteriostatic, bactericidal, fungistatic, fungicidal, antiparasitic and/or antiviral activity.

The term “free iron” refers to iron which is not bound to a chelator or carrier protein prior to use in a method or a composition of the present invention. “Low concentrations” of free iron refers to concentrations of iron less than about 1 M iron, optionally ranging from, for example, about 0.01 μM to about 100 mM, about 0.01 μM to about 1 mM, about 0.1 μM to about 10 mM, or about 0.1 μM to about 1 mM free iron. Optionally, the “low concentrations” of free iron are concentrations of free iron that sensitize the cell such that a five-fold lower concentration (or intensity or duration) of stress inducer is required for microbiocidal activity, as compared to when the stress inducer is used alone as a disinfectant.

The term “stress inducer” refers to a compound or environmental parameter that “stresses” a biological system, induces a stress response, or otherwise alters the biological functioning of a cell or organism. “Oxidant” stress inducers are compounds or environmental parameters that function, at least in an initial step, via the generation of an oxygen free radical. “Non-oxidant” stress inducers are compounds or environmental parameters that, in an initial step, do not involve an oxygen free radical.

The term “organic acids” refer to any of a number of carbon-containing acid compounds, such as acetic, citric, tartaric, or mandelic acid. The term “inorganic acids” therefore refers to non-carbon containing acidic compounds, such as hydrochloric acid and nitric acid.

Methods Of Reducing Microbial Populations

The present invention provides novel methods for reducing a microbial population on a surface. In one embodiment, the methods of the present invention include the steps of a) providing a preparation of free iron; b) providing a stress inducer; and c) bringing the surface into contact with the preparation of free iron and the stress inducer, thereby reducing the microbial population on the surface. In another embodiment, the methods of the present invention include the steps of a) providing a preparation of free iron; b) providing a non-oxidant stress inducer; and c) bringing the surface into contact with the preparation of free iron and the non-oxidant stress inducer, thereby reducing the microbial population on the surface. In a further embodiment, the methods of the present invention include the steps of a) providing a low concentration of free iron; b) providing a stress inducer; preferably a non-oxidant stress inducer, and c) bringing the surface into contact with the low concentration of free iron and the non-oxidant stress inducer, thereby reducing the microbial population on the surface. The common element of these methods is the use of varying concentrations of free iron, in conjunction with a stress inducer (preferably, a non-oxidant stress inducer).

Transition metals such as iron, zinc, manganese and copper are necessary for microbial cell growth. However, in their ordinary environmental niches, such as the soil, an intestinal lumen, an animal's bloodstream, a skin surface, or an inorganic surface such as a cutting board or a medical instrument, microbes are relatively starved for transition metals like iron. One reason for this is that, using iron as an example, the soluble, reduced form of iron (Fe²⁺) spontaneously oxidizes to the relatively insoluble reduced form of iron (Fe³⁺), making the element less accessible. Furthermore, many higher organisms have mechanisms in place for sequestering transition metals (for example, transferrin), also making them unavailable for microbial use. In response, bacteria have developed a variety of mechanisms, albeit elaborate and energetically expensive, for absorbing these essential elements from the environment.

Since iron and other transition metals enable and enhance bacterial growth (i.e., they are pro-microbial), these elements have not been used as antimicrobial compounds. Rather, some of the most well-studied bacteriostatic compositions currently employed direct the removal, not the addition, of iron from the environment, thus inhibiting microbial growth. Examples of these bacteriostatic compositions include transferrin, a protein in eukaryotic cells which binds iron and renders it unavailable to bacteria, and the chelating agent ethylenediamine tetraacetic acid (EDTA, a widely used antimicrobial preservative). Furthermore, conditions which increase iron availability in animals (such as the human iron overload disease hemochromatosis) tend to enhance bacterial growth and dramatically worsen bacterial infections. All of these considerations have led to the conclusion that essential transition elements such as iron are pro-microbial.

Despite their growth enhancement capabilities, transition metals can also be toxic to both eukaryotic and bacterial cells under certain circumstances. For example, reduced (ferrous, Fe²⁺ or Fe(II)) iron, together with the oxidant hydrogen peroxide, form the highly reactive hydroxyl radical (·OH) in a reaction commonly described as Fenton chemistry (See, for example, Mar., ed. Advanced Organic Chemistry: Reactions, Mechanisms and Structure Third Edition (1985) John Wiley and Sons, New York).

In the presence of a reducing agent such as ascorbate, the transition metals can function as catalysts, first being oxidized by the peroxide oxidant (and forming the damaging hydroxyl radical, as shown above), then being returned to their original reduced state by reacting with the ascorbate reducing agent. The hydrogen peroxide and ascorbate are consumed in the reaction, producing hydroxyl radicals and regenerating the ferric iron, in a series of reactions know as iron- or copper- catalyzed Haber-Weiss reactions:

Half reactions:

| | | |
|---|---|---|
| Fe(II) + H₂O₂ | → | Fe(III) + •OH + OH⁻ |
| FE(III) + Ascorbate (red) | → | Fe(II) + Ascorbate (ox) |

Net reaction:

| | | |
|---|---|---|
| Ascorbate (red) + H₂O₂ | → | Ascorbate (ox) + •OH + OH⁻ |

The few examples in the art in which transition metals have been used as disinfectants have generally also employed one or more oxidants (leading to the previously described Fenton reaction), or the transition metal was provided in a mixture with a peroxide and a reducing agent (i.e., the Haber-Weiss reaction). Such long-term, high-dose treatments have the two major disadvantages. First, long term treatments are too lengthy to be incorporated into some processed in which microbial populations should be reduced, such as automated food-processing operations. Furthermore, the high-dose treatments leave undesirable residues on the treated surface.

The novel methods of the present invention reduce microbial populations in part by causing a “transition metal overload” (TMO) within the microbial cell (i.e., he transition metal is internalized so rapidly that the existing cellular mechanisms for neutralizing these redox-reactive metals are overwhelmed). The present invention overcomes the limitations in the art by providing methods in which the process of reducing microbial populations can optionally be performed in surprisingly short periods of time, for example, one the order of minutes instead of days. In addition, the present invention optionally employs low doses of free iron (for example, on the order of 100 micromolar or less). The present invention provides novel methods of reducing microbial populations through exposure of a surface to transition metals such as free iron, as well as the use of the free iron to sensitize microbes to a wide range of stress inducers (e.g., physical and chemical disinfectants, and the like).

Preparations Of Transition Metals

The methods of the present invention are performed by exposing microbial cells to one or more bioavailable transition metals. Unlike multicellular eukaryotic organisms, microbial cells are unable to handle a sudden excess in transition metal(s) in the environment. The resulting increase in intracellular concentration of the transition metal(s) promotes oxidative damage, thus weakening the cells and sensitizing the cellular response to other oxidative or non-oxidative challenges. Preferably the transition metal(s) used are Generally Regarded as Safe (GRAS) compounds, and as such do not cause harm to the host cells upon exposure to the transition metal used in the method.

The particular bioavailable transition metal(s) used will depend to some extent upon the specific bacterial species in question and the specific metal transport proteins associated with the species. Transition metals include the elements chromium, manganese, iron, nickel, copper, zinc, and molybdenum, to name a few. The bioavailable forms of transition metals include, but are not limited to, various salts or organic complexes of the reduced and/or oxidized transition metal, such as acetate, ammonium citrate, ammonium oxalate, ammonium sulfate, bromide, chloride, citrate, fluoride, fumarate, hydroxide, iodide, nitrate, oxide, phosphate, pyrophosphate, sulfate, and tartrate. One or more of the transition metal derivatives can be prepared in a suitable formulation for the intended use. Optionally, the transition metal formulation is prepared in water or in hypo-osmotic saline solutions.

Of particular interest are reduced forms of iron (Fe(II)) and copper (Cu(II)). Most microorganisms absorb ferrous and cuprous ions via specific transport proteins embedded in their membranes. Both iron and copper are GRAS compounds at the highest potential residue levels that might be achieved in the present invention. Also of interest is heme-bound iron because some species (e.g., Cainpylobacter) or strains (e.g., E. coli O157:H7) appear to preferentially absorb heme-bound iron.

A range of concentrations of the transition metal (for example, free iron) can be employed in the methods of the present invention. For example, the low concentration of free iron can range between about 0.01 μM and about 100 mM free iron, optionally between about 0.1 μM and about 1 mM free iron, or between about 1 μM and about 1 mM free iron. Preferably, the concentration of free iron used in the method is approximately 1, μM free iron. The optimum concentrations and exposure duration to achieve sufficient increases in intracellular transition metals to significantly decrease viability for different microbial species can be determined empirically for each species of interest using methods known to those of skill in the art for evaluating microbiocidal activity. Optionally, the bioavailable transition metals tested will be those for which the particular organism is know to have a specific transport system.

Generally, when a more rapid the treatment is desired, a higher concentration of transition metal is employed in the method, to achieve a desired degree of disinfection (e.g., about 90%, about 95%, about 98%, about 99%, about 99.9%, or greater reduction in viable microbial count). Similarly, more rapid treatments optionally entail the use of higher concentrations/durations/intensities of stress inducer. High throughput methods, as disclosed herein as well as those known to one of skill in the art, are used to provide multifactorial data sets from hundreds of combinations of compositions, concentrations, durations, and the like.

Oxidant And Non-Oxidant Stress Inducers

A number of stress inducers can be employed in the method of the present invention. “Stress inducers” are compounds or environmental parameters that induce a stress response in a biological system, or otherwise alter the biological functioning of a cell or organism. The stress inducers preferably act synergistically with the bioavailable transition metal(s) to provide for enhanced microbiocidal activity, for example, by taking advantage of the increased sensitivity of the microorganisms after transition metal overload.

An example of a stress inducer is a chemical disinfectant, or antiseptic. Chemical disinfectants generally fall into broad categories such as halides (e.g. iodine and iodophores, bromine, chlorine and chlorine-based products), organic solvents (e.g. methanol, ethanol, isopropanol, phenol, xylenol), organic and inorganic acids (e.g. lactic acid, acetic acid, hydrochloric acid, nitric acid, formic acid, and the acid forms of biological molecules such as propionate, pyruvate, and succinate), reactive nitrogen species (e.g., nitrites, nitrates, nitric oxide, peroxynitrite, and the like), bases (e.g. trisodium phosphate, various quaternary amines such as cetylpyridinium chloride), chelators (e.g., EDTA), and aldehydes (e.g., formaldehyde, glutaraldehyde, acetaldehyde). Additional chemical disinfectants include, but are not limited to, oxidants (e.g. peroxides, hypochlorite, ozone), sorbates, as well as soaps, detergents and other surfactants.

The stress inducers of the present invention also include physical disinfectants (i.e., disinfectants that operate via changes to one or more environmental parameters). Examples of physical disinfectants include, but are not limited to, irradiation (e.g. ultraviolet or ionizing), heat (e.g., steam and liquid-phase pasteurization), osmotic shock, ultrasonic disruption, pressure, freezing, changes in pH, and the like. Further stress inducers related to changes in environment parameter include, but are not limited to, various hypo-osmotic solutions, hyper-osmotic solutions, and the like are also considered in some embodiments of the present invention.

In some embodiments of the present invention, the stress inducer is a non-oxidant stress inducer. Non-oxidant stress inducers are compounds or environmental parameters that, in an initial step, do not involve generation of an oxygen free radical. This is in contrast to “oxidant” stress inducers, which are compounds or environmental parameters that generate an oxygen free radical as a first reaction step. Many of the chemical and physical stress inducers described above can be considered non-oxidizing stress inducers. Additional examples of chemical disinfectants that are considered to be “non-oxidant stress inducers” for the purposes of the present invention include antibiotics, antiseptics, antimicrobial agents, and lytic enzymes, such as lysozyme, bromelain, papain, peptidases, lipases, amylases, carboxylases, carrageenans and other sulfated polysaccharides, cell wall degrading enzymes, (e.g., cellulase, endoxylanase, invertase, lactamase, pectinase, zymolase) and the like. Furthermore, metal-chelating polymers, such as polyamines, chitin, and chitosan, can be employed as non-oxidizing stress inducers in the methods of the present invention. Other polymers are also considered, including, but not limited to, celluloses, carboxymethylcelluloses, cyclodextrans, fucoidans, gellans, histones, mucopolysaccharides, pectins, polyacrylates, polyamino acids (e.g., polylysine), polycarboxylates, polyglucosamines, polyisobutylenes, polymannans (such as β(1→4) acetylated mannans, or “aloe”), polyvinylalcohols, polyvinylsaccharides, polysialic acids, polyurethanes, protamine, scleroglucans, starches, xanthans, and other natural and synthetic polymers. Polymers for use as stress inducers of the present invention optionally form films on the surface being treated; said films are optionally water-absorbent or water resistant. The polymers are capable of inducing stress upon the organism being treated, as well as optionally providing a convenient carrier (but not necessarily as a chelator) for the transition metal preparation during treatment of the surface.

One preferred group of non-oxidant stress inducers are chitin, chitosan, and other chitin derivatives. Chitin is a polymer of β(1→4)-linked N-acetylglucosamine, found in the shells of crustaceans such as crabs and shrimp. The related polymer chitosan is a deacetylated form of chitin. Chitin and chitosan have been shown to have antimicrobial activity, as well as an acceleratory effect on wound healing. In some embodiments of the present invention, non-oxidant stress inducers such as chitin and chitosan are considered. In other embodiments, non-oxidant stress inducers other than chitin, chitosan, and other chelating polymers are contemplated.

A range of concentrations of stress inducer are considered in the methods of the present invention. One of skill in the art will appreciate that the concentration of stress inducer employed in the methods will vary, depending upon, for example, the molecular weight ranges of the stress inducer employed, the nature of the surface being treated, and the microbial organisms to be affected. The relative amounts of transition metal and stress inducer can easily be determined empirically, using techniques known in the art. Furthermore, the stress inducer can be contacted with the surface to be treated concomitant with the transition metal preparation, after the surface has been contacted with the transition metal preparation, or even before the surface has been contacted with the transition metal preparation.

Treatment Schemes

The step of bringing the surface of choice into contact with the free iron preparation (optionally, a low concentration of free iron) and the stress inducer can be performed in any of a number of ways. For example, the free iron preparation can be applied separately from the stress inducer, the two compositions can be coadministered, or the two compositions can be combined prior to application to the surface. For instance, the surface can be sprayed (with solutions of free iron and/or stress inducer) or dusted (with powder forms of free iron and/or stress inducer). Alternatively, the surface can be either completely or partially submerged, or “dipped,” into the free iron and/or stress inducer preparations. The antimicrobial preparations can be applied with a sponge, a mop, a cloth, or any other of a variety of techniques known to one of skill in the art of disinfection. Furthermore, combinations of these application techniques can be employed (for example, the surface may be submerged in the free iron preparation, and subsequently sprayed with the stress inducer preparation).

In one embodiment of the methods of the present invention, a portion of the free iron is removed prior to administration of the stress inducer. Approximately 50%, 75%, 90%, 95%, 98%, 99%, 99.5%, or substantially all of the preparation of free iron can be removed prior to bringing the surface into contact with the stress inducer. The free iron preparation can be removed in a number of ways, including, but not limited to, rinsing, wiping, washing, scrubbing, precipitating, and the like.

Optionally, the surface is exposed to one or more of the free iron preparation and/or the stress inducer preparation for a transient length of time suitable to reduce the microbial population on the surface. Suitable lengths of time will depend, in part, upon the surface to be treated, as discussed in greater detail in the following section. For example, a suitable length of time for exposure of the surface to one or more of the free iron preparation and the stress inducer range from 6 seconds to one week, depending upon the application. Exemplary lengths of time include, about 6 seconds, about 15 seconds, about 30 seconds, about 3 minutes, about 1 hour, about 4 hours, about 12 hours, about 24 hours, and about 1 week.

For effective disinfection, it is desirable to apply a known concentration of disinfectant (e.g., free iron and/or stress inducer) to the surface to be treated for a determined period of time. Adhesion of the disinfectant preparation to the surface can be enhanced by addition of adhesion agents, thickening agents, foaming agents, and the like. Furthermore, the methods of the present invention can optionally employ equipment such as nebulizers, which result in foaming, or other compounds and procedures known to those skilled in the art which result in a foam or viscous gel to ensure adequate exposure of the disinfectant solutions to the treated surface. The foam or gel, by adhering to the surface of, for example, a food product, provides a longer exposure time than would be achieved with a simple liquid.

The transition metal preparation can also be applied to the surface to be treated as a dispersed powder. For example, a poultry carcass, wet from de-feathering, could be passed through a device in which a fine powder of the transition metal preparation (e.g., the free iron preparation) is continuously circulated by air blowers, in such a way that the carcass was evenly coated with a layer of the powder. The free iron preparation dissolves in the aqueous surface film on the bird. A similar method could be used to dust dry or wetted produce of any type. The benefit of using iron or copper dusts is that once these dusts are solubilized, the local concentration of iron or copper is very high. Another benefit would be that such a method of application would likely be economical from the standpoint of consuming very little of the iron or copper compounds. Additional benefits of using powders include the stability of most chemicals in dry form versus in solutions (facilitating the shipping and storage of the preparations), and the fact that no extra water would be required during the application process. Such powders could moreover include other agents, such as detergents, emulsifiers, and other chemical co-disinfectants, and the dry and solubilized residues could be removed from the surfaces of foods by a simple rinse.

Organisms

The methods of the present invention take advantage of a key difference in the mechanisms for metabolizing transition metals between rapidly-dividing single celled organisms (such as pathogenic bacteria) and metazoans (such as humans). As an example, microorganisms and mammals metabolize iron using different mechanisms. Iron ions commonly exists in two prevalent redox states, the relatively soluble ferrous iron (Fe(II) or Fe²⁺) and relatively insoluble ferric iron (Fe(III) or Fe³⁺). Since Fe²⁺ is spontaneously oxidized to Fe³⁺ in an aerobic environment, there is little Fe²⁺ available in most natural environments, and hence there is generally little iron or copper available for rapidly dividing microbes. Furthermore, a key defense of many multicellular organisms against bacterial contamination is the chelation of transition metals in forms that are unavailable to bacteria. Divalent chelators such as ethylenediamine tetraacetic acid (EDTA) are broadly used as food preservatives, because they prevent bacterial growth by starving microbes of essential iron. The methods of the present invention take advantage of the observation that microorganisms appear to rapidly take up any iron that is made available, even if such a dose will prove lethal. The methods of the present invention are also effective against viral populations.

Microbial population which are susceptible to the methods and or compositions of the present invention include prokaryotic organisms, fungi and molds, and yeast. Exemplary prokaryotic organisms include, but are not limited to, Bacillus, Burkholderia, Campylobacter, Chlamydia, Clostridium, Corynebacterium, Escherichia, Hemophilus (Haemophillus), Helicobacter, Legionella, Listeria, Mycobacterium, Mycoplasma, Neisseria (Meningococcus), Pseudomonas, Salmonella, Shigella, Staphylococcus, Streptococcus, Trypanosoma, Vibrio, and Yersinia (See, for example, the lists of microorganism genera provided by DSMZ-Deutsche Sammlung von Mikroorganismen und Zellkulturen GmbH, Braunschweig, Germany, at http://www.dsmz.de/species, and the Center for Disease Control website, http://www.cdc.gov). Exemplary fungi, molds and yeast which can be treated using the methods compositions and/or kits of the present invention include, but are not limited to, Actinomycetes, Aphanomyces, Aspergillus, Botrytis, Candida Cladosporium, Cryptococcus, Fusarium, Malassezia, Mucor, Neurospora, Penicillium, Rhizobium, Rhyzoctonia, Rhizopus, ringworm fungi (e.g., Microsporum, Epidermophyton and Trichophyton), Saccharomyces, and Streptomyces. Additional unicellular and/or parasitic organisms which can be reduced in population using the methods of the present invention include, but are not limited to, various algaes, slime molds, and water molds, as well as parasitic organisms such as Cryptosporidia, Giardia, Plasmodium, Toxoplamsa.

The methods of the present invention can also be employed to reduce one or more viral population on a surface. Viruses which are susceptible to the methods and compositions of the present invention include, but are not limited to, various strains of rotovirus; adenoviruses; herpes viruses; variola, vaccinia and other pox viruses; polio and other picorna viruses (including enteroviruses and rhinoviruses); calcivirus; coronaviruses; hepatitis; influenza; rhabdoviruses (rabies); rubella and other togaviruses; papova viruses such as SV40, polyoma and papilloma viruses; various oncogenic viruses (Epstein-Barr virus, herpes simplex virus, cytomegalovirus); and the like. For a general review, see Dulbecco and Ginsberg Virology (reprinted from Davis, Dulbecco, Eisen and Ginsberg's Microbiology, third edition (1980) Harper and Row, Philadelphia, Pa.).

Treatable Surfaces

The methods of the present invention provides novel methods by which a microbial population on a surface can be reduced or eliminated. The surface to be treated can be composed of, for example, metal, ceramic, wood, glass, plastic, rubber, and the like. Items including, but not limited to, kitchen utensils and work surfaces, bathroom fixtures and floors, industrial equipment, and medical equipment and surfaces are contemplated as surfaces to be treated using the methods and compositions of the present invention. Additionally, biological surfaces such as intact epidermal surfaces, damaged tissues, and infected surfaces (e.g., regions affected by infections such as acne, athlete's foot, vaginal yeast infections and the like) are also contemplated as surfaces to be treated using the methods and compositions of the present invention. In some embodiments, the methods involve contacting a surface to be treated with a preparation of free iron, and a stress inducer (optionally, a non-oxidant stress inducer). In alternate embodiments, the methods include contacting a surface to be treated with a preparation of free iron and one or more siderophores. In further embodiments, the methods include contacting a surface to be treated with a preparation of free iron and one or more polymers or polysaccharides. The contacting can be performed in any of a number of ways, or in a combination of ways, as desired by one practicing the method. The methods described herein are generally applicable to any of a number of surfaces, a few examples of which are discussed below.

Decontamination of Food

In one embodiment of the present invention, the surface to be reduced in microbial population includes a food surface. Spoilage of food products, as well as potential infection following ingestion of contaminated food products, can be reduced by treating food surfaces using the methods of the present invention. Any number of food surfaces can be treated by the methods of the present invention, particularly since the transition metal compositions are considered to be “generally regarded as safe.” Exemplary food surfaces include, but are not limited to, meat and poultry carcasses and parts, fish and shellfish, eggs, nuts, and fresh and processed vegetable and fruit produce (e.g. field harvested crops such as lettuce; hothouse-grown produce such as alfalfa sprouts; and picked fruit such as oranges, apples, grapes, and berries).

The present invention provides methods for reducing a microbial population on a food surface. The methods include the steps of a) providing a composition of a transition metal, such as iron; b) providing a non-oxidant stress inducer; and c) bringing the food surface into contact with the free iron and the non-oxidant stress inducer, thereby reducing the microbial population on the food surface. While iron is specifically addressed in this example of the methods of the present invention, any number of transition metals can be employed Two particularly preferred transition metals are iron and copper. The transition metal (or combination of transition metals) is provided, for example, as a salt, an organic complex, or a combination thereof. Exemplary salts and/or organic complexes include, but are not limited to, acetate, ammonium citrate, ammonium oxalate, ammonium sulfate, bromide, chloride, citrate, fluoride, fumarate, hydroxide, iodide, nitrate, oxide, phosphate, pyrophosphate, sulfate, and tartrate. The metal salt and/or organic complex can be prepared in water, or it can be prepared in a formulation (e.g., a buffer) suitable for the intended use. Optionally, the iron formulation is prepared in water or a hypo-osmotic saline solution.

The transition metal can also be combined with one or more oxidant or non-oxidant stress inducers. Preferred stress inducers for use in the methods of decontaminating a food surface include, but are not limited to, the oxidant stress inducers H₂O₂ and bleach, and the non-oxidant stress inducers lysozyme and other lytic enzymes (e.g., peptidases, carboxylases, nucleases, cell wall degrading enzymes, and the like); soap, detergent, and other surfactants; polymers such as polysaccharides, adhesive polymers, and the like; and chelating polymers (such as polyamines, chitin, chitosan). However, other stress inducers, such as physical disinfectants (e.g., irradiation, osmotic shock, etc.), can be employed.

Contacting the food surface with the free iron and stress inducer can be accomplished by any of a number of techniques as described previously, including spraying and dipping. Furthermore, the surfaces can be treated, for example, either prior to harvesting, or post-harvest. Optionally, contacting the food surface includes forming a protective film on the food surface, which can later be either ingested or removed.

Furthermore, a range of concentrations of free iron can be employed in the methods for reducing a microbial population on a food surface. For example, the free iron can be provided at a concentration as low as between about 0.1 μM and about 1 M free iron. Optionally, the concentration of free iron is between about 0.1 μM and about 100 mM free iron, preferably, 0.1 μM and about 1 mM free iron, and more preferably about 1 μM free iron. Higher concentrations of iron and or copper and stress inducer may also be of particular use in some embodiments, in which microbes are protected from applied solutions or powders by sequestration inside crevices on the surface of the food product (e.g. in the feather follicles of a poultry carcass, between muscle fibers on beef) or by being sheltered by layers of fat, grease, wax, or any other matter which reduces the contact between externally applied compounds and the microbes themselves (e.g. on the greasy surface of a poultry carcass, in associated with the waxy cuticle on fruit). In such circumstances, treatment of bacteria with higher concentrations of iron or copper (e.g. from 1 mM to 1 M) may be a simple way to achieve effective concentrations (e.g. 10 μM to 1 mM) in the fluid immediately surrounding the microbes themselves.

The food surface to be treated is exposed to the free iron and/or stress inducer for a length of time sufficient to decrease the microbial population. This sufficient length of time is easily determined by one practicing the methods of the present invention. For example, the food surface can be exposed for as short a time period as 5 seconds, or the amount of time that it would take to dip the food article into a preparation of the free iron. Alternatively, the free iron can be applied to the food surface and left there (i.e., not specifically removed or washed away after treatment of the surface). Times of exposure between these two extremes are also contemplated in the methods of the present invention, as described in greater detail in previous sections. The free iron and the stress inducer can be coadministered, or they can be applied to the surface sequentially. Optionally, the free iron can be applied first, and subsequently removed prior to bringing the surface into contact with the stress inducer. In this embodiment, for example, approximately 50%, 75%, 90%, 95%, 98%, 99%, 99.5%, or substantially all of the free iron is optionally removed. The optimum concentrations and exposure durations can be determined empirically for the food surface to be treated (e.g., taking into account the type of food surface, physical characteristics of the surface (e.g., porosity), and extent of exposure the food surface can withstand). Furthermore, the species (or groups of species) to be reduced in population can play a role in determining the treatment parameters, using methods for evaluating microbiocidal activity known to one of skill in the art.

An example of the type of considerations that will result in the selection of high concentrations (in concert with shorter exposures) versus low concentrations (and longer exposures) is the processing of poultry. In this process, birds are hung on shackles and move along a conveyer through the factory. The transit time generally involves less than 15 minutes from the time of slaughter to the time that carcasses are deposited into a chiller water bath, in which they spend roughly 45 minutes before final packaging. There are numerous points in the processing plant at which the stress inducers may be applied. To give just two contrasting examples, stress inducers can be applied early on in the plant at the time of defeathering, a point in the process at which spreading of microbes is most likely. Stress inducers can also (or alternatively) be included in chiller water itself, as is currently the case with chlorine. Defeathering is a rapid process; birds pass through the device in a matter of 15-30 seconds or so, and is followed by many other processing steps, including numerous water rinses, before the carcasses are deposited in a water chiller. In contrast, the chiller bath is a long treatment, and is the final rinse for the carcasses, which are then immediately packaged; as a consequence, residue from the chiller itself ultimately ends up in packaged products. Therefore, high concentrations of iron or copper (e.g. 100 mM Fe²⁺, for example) may be preferable during the brief defeathering procedure, since these high concentrations will later be removed by rinsing, and since high concentrations will have a greater effect during such a short period of treatment. In contrast, far lower concentrations (10 μM or lower) may be preferable during a long chiller bath exposure, since in this case the solution may end up on the final product, where high concentrations may result in staining or a metallic taste. Therefore, specific embodiments include all those treatments, which are determined empirically by the use of high-throughput methods such as those disclosed herein, in which either rapid exposures to iron- and/or copper- based composition or low concentrations or such compositions are found to be effective in decontaminating said food items.

Buffered acidic solutions, such as may be achieved with weak organic acids such as acetic, citric, tartaric, or mandelic, are optionally used to prepare the transition metal preparation, since the solubility of iron and copper are increased at acidic pH, and since an acidic pH can also function as a stress inducer. The exact pH and acid used will depend to some extent on the suitability of acid conditions to the application in question. A large number of ligands are also known, to those skilled in the art, for the solubilization of iron, e.g. citrate, fumarate. It may also be particularly useful for solutions to be hypo-osmotic (e.g. prepared in pure water), since it is disclosed here that iron or copper overload sensitizes microbial cells to the stress of hypo-osmotic shock. Since reduced iron and copper in solution are subject to oxidation and subsequent precipitation, compositions requiring the reduced form of the transition metals preferably include powdered preparations that are stored in dry form and solubilized immediately prior to use.

Many food products possess complex surfaces involving barriers of grease, oils, or waxes under which pathogenic bacteria may be sequestered. These complex surfaces can also interfere with the contact of transition metal and/or and stress inducer solutions with target microbes. Consequently, formulations for use in the methods of the present invention optionally include one or more solvents, soaps, emulsions, detergents, foaming agents, or other means known to those skilled in the art for breaking down hydrophobic films. Optionally, physical methods such as the use of high-pressure sprays, sonication, or heated solutions are used to reduce and/or permeabilize water-impermeable layers. Contaminating bacteria are also often found in crevices and pores on foods, which can be an obstacle to the delivery of disinfectant compositions. Hence techniques for delivering formulations to such pores and crevices (e.g., high-pressure sprays, sonication, surfactants, and other means known to those skilled in the art) are optionally employed in the methods of the present invention.

Although the ingestion of moderate amounts of copper and large amounts of iron over an extended period of time may result in health problems in humans, the residues on food from the disclosed compositions and methods pose no health risk to consumers. Indeed, both iron and copper are essential for human health, and the average American typically consumes milligram quantities of iron and copper each day in food and/or dietary supplements. The safety of iron is particularly evident, since the U.S. recommended daily allowance is 18 mg (and human infant formula is routinely supplemented with even larger amounts of the element). In preferred embodiments of the present invention, the amount if iron remaining on treated surfaces of decontaminated foods will generally be far lower than the amount of iron or copper in the food itself. It is therefore not essential to remove iron or copper residues from decontaminated foods prior to packaging. However, since the residual iron may impart an unpleasant flavor, or may leave a yellow, brown, or black colored deposit upon the treated surface, the iron preparation can optionally be removed prior to packaging or consumption.

Medical and Veterinary Applications

In one embodiment of the present invention, the surface to be reduced in microbial population includes a living tissue (e.g., a plurality of host cells). The plurality of host cells includes an intact surface, such as an epidermal surface (e.g., the skin of a patient or the teat of a diary cow), as well as damaged tissues, such as a wound, an abrasion, a burn and the like. Other living tissues include, but are not limited to, the surfaces lining an oral cavity, a vaginal cavity, an intestinal tract, and the like (e.g., mucosal surfaces). Host cells have specific systems to manage exposure to changes in transition metals in the environment. For example, in higher organisms such as man, iron and other transition metals are present in a bound, or “protected,” form (either in carrier/storage proteins such as ferritin, lactoferrin and transferrin, or in the active site of redox proteins such as catalase), and as such are not capable of catalyzing destructive reactions and generally wreaking havoc in the cell. However, bacteria and other microbes do not have mechanisms for dealing with excess transmission metals. The methods of the present invention take advantage of the ability of the plurality of host cells (for example, a skin surface of a human) to manage “transition metal overload” better than microbial cells.

The skin is the first line of defense in protecting one's body from microbial infection. The outermost surface of the skin is comprised of squamous epithelial cells, which are continuously being removed (sloughed off) as new cell growth occurs. Damaged skin (e.g., a wound, an abrasion, a bum, or a damaged region of tissue) provides a portal through which invading microbes can potentially enter. The methods and compositions of the present invention can be used to reduce the populations of microbes in these regions, thus reducing the possibility of infection.

Disinfectants commonly used for the cleaning of epidermal surfaces and/or damaged tissues such as wounds are typically non-selective. While effective, iodine, hydrogen peroxide, soap, and other disinfectants often destroy host cells along with pathogens. Multicellular organisms (such as humans) can tolerate the killing of a certain percentage of “host cells” in a wound, which is preferable to the unchecked growth of bacterial pathogens. However, since the ultimate defense of the host against infection is due to its immune system, the destruction of immune cells along with bacteria is less than an ideal solution.

The use of antibiotic-containing creams, which selectively target microbial processes and kill susceptible cells but generally are benign to the host at doses to which susceptible bacteria are sensitive has become a common first aid technique. Antibiotics, such as are ingested by humans with systemic infections, or spread topically across areas to be treated, typically work by targeting metabolic processes which are unique to bacteria, and are therefore innocuous to host cells. Antibiotics are hence distinct from disinfectants (antiseptics), both because antibiotics are generally harmless to the host, and because they target a single or a small number of metabolic processes. However, antibiotic-resistant strains of bacteria arise which overcome the specific modes of action of antibiotics. In other words, the strength of antibiotics—that they target particular and unique bacterial molecules —is also a weakness, since mutation of the genes encoding antibiotic targets may render a bacterium capable of overcoming the antibiotics. In this regard, the strength of disinfectants such as hydrogen peroxide is that they attack a broad-spectrum of molecules and thereby defy attempts by pathogens to evolve resistance.

The present invention provides methods for reducing a microbial population on a living tissue. The methods include the steps of a) providing a composition of free iron; b) providing a stress inducer (optionally, a non-oxidant stress inducer); and c) bringing the living tissue into contact with the free iron and the stress inducer, thereby reducing the microbial population on the living tissue. These methods have the advantage of selectively harming the microbial cells while not interfering with host cell growth (similar to an antibiotic) while retaining the ability to affect more than one type of microbe (similar to a disinfectant), thus providing novel mechanism to reduce the population of microbes on a living tissue.

Any number of transition metals can be employed in the methods of the present invention, but a particularly preferred transition metal is iron. The transition metal (or combination of transition metals) is provided, for example, as a salt, an organic complex, or a combination thereof. Exemplary salts and/or organic complexes include, but are not limited to, acetate, ammonium citrate, ammonium oxalate, ammonium sulfate, bromide, chloride, citrate, fluoride, fumarate, hydroxide, iodide, nitrate, oxide, phosphate, pyrophosphate, sulfate, and tartrate. The metal salt and/or organic complex can be prepared in water, or it can be prepared in a formulation (e.g., a buffer) suitable for the intended use. Optionally, the iron formulation is prepared in water or a hypo-osmotic saline solution.

The transition metal can also be combined with one or more oxidant or non-oxidant stress inducers. Preferred stress inducers for use in the methods of treating a living tissue include, but are not limited to, halides (e.g., iodine), organic solvents such as ethanol, the oxidant stress inducer H₂O₂, and the non-oxidant stress inducers lysozyme (and other lytic enzymes), polysaccharides (e.g., chitin, chitosan), soap, detergent, and other surfactants. Optionally, other stress inducers, such as physical disinfectants (e.g., osmotic shock), can also be employed, either as the primary stress inducer or in conjunction with an additional stress inducer.

The surfaces can be treated, for example, prior to contact with a living tissue (e.g., before milking a cow), prior to an invasive process (e.g., before a surgical procedure), or after the living tissue has been damaged (e.g., as part of a treatment regime for a wound). Contacting the living tissue with the free iron and stress inducer can be accomplished by any of a number of techniques as described previously, including spraying and dipping. Alternatively, the free iron and stress inducer can be applied to a matrix or other medium for transporting and/or retaining the preparation against the surface to be treated. For example, the preparation can be applied to a mop, a sponge, a cloth, a bandage, a gauze, or other material to be placed against a wound. One of skill in the art would note that the sponges or gauzes used for such a medical application differ in their synthesis and chemical composition from a sponge or cloth used for another, non-medical application (such as cleaning a countertop).

A range of concentrations of free iron can be employed in the methods for reducing a microbial population on a living tissue. For example, the free iron can be provided at a concentration as low as about 0.1 μM to as high as about 1 M free iron. Optionally, the concentration of free iron is between about 0.1 μM and about 100 mM free iron, preferably about 1 μM to 10μM free iron. The living tissue to be treated is exposed to the free iron and/or stress inducer for a length of time sufficient to decrease the microbial population. This sufficient length of time is easily determined by one practicing the methods of the present invention. For example, the living tissue can be exposed for as short a time period as 5 seconds, or the amount of time that it would take to swipe a skin surface or a cow teat with a preparation of the free iron. Alternatively, the free iron can be applied to the living tissue and left there (i.e., not specifically removed or washed away after treatment of the surface). Times of exposure between these two extremes are also contemplated in the methods of the present invention, as described in greater detail in previous sections. The free iron and the stress inducer can be coadministered, or they can be applied to the surface sequentially. Optionally, the free iron can be applied first, and subsequently removed prior to bringing the surface into contact with the stress inducer. In this embodiment, for example, approximately 50%, 75%, 90%, 95%, 98%, 99%, 99.5%, or substantially all of the free iron is optionally removed. The optimum concentrations and exposure durations can be determined empirically for the living tissue to be treated (e.g., taking into account the type of living tissue, physical characteristics of the surface (e.g., whether it is damaged, extent of damage), and extent of exposure the living tissue can withstand). Furthermore, the species (or groups of species) to be reduced in population can play a role in determining the treatment parameters, using methods for evaluating microbiocidal activity known to one of skill in the art.

Decontamination of Water

In another embodiment of the present invention, the “surface” to be reduced in microbial population includes an aqueous solution. While not considered a typical surface, the aqueous solution can be reduced in microbial population using the methods of the present invention.

The present invention provides methods for reducing a microbial population in an aqueous solution. The methods include the steps of a) providing a composition of free iron; b) providing a stress inducer, optionally a non-oxidant stress inducer; and c) bringing the aqueous solution into contact with the free iron and the stress inducer, thereby reducing the microbial population in the aqueous solution. Any number of transition metals can be employed in the methods of the present invention, including, but not limited to, iron, copper, vanadium, chromium, manganese, nickel, zinc. The transition metal (or combination of transition metals) is provided, for example, as a salt, an organic complex, or a combination thereof. Exemplary salts and/or organic complexes include, but are not limited to, acetate, ammonium citrate, ammonium oxalate, ammonium sulfate, bromide, chloride, citrate, fluoride, fumarate, hydroxide, iodide, nitrate, oxide, phosphate, pyrophosphate, sulfate, and tartrate.

The transition metal can also be combined with one or more oxidant or non-oxidant stress inducers. Preferred stress inducers for use in the methods of decontaminating an aqueous solution include, but are not limited to, the non-oxidant stress inducer lysozyme, and physical disinfectants (e.g., irradiation, heating, freezing, changes in pressure).

Contacting the aqueous solution with the free iron and stress inducer can be accomplished by any of a number of techniques as described previously, including spraying the free iron preparation over the aqueous solution, and passing the aqueous solution through a matrix or a filter containing the free iron preparation, the stress inducer, or a combination thereof. For example, the free iron preparation is added to the aqueous solution, and the mixture is then passed through a column containing the non-oxidant stress inducer lysozyme bound to a column matrix composition. Optionally, the free iron preparation can be removed from the aqueous solution, either prior to when the stress inducer has been provided, or after the treatment has been completed.

Furthermore, a range of concentrations of free iron can be employed in the methods for reducing a microbial population within an aqueous solution. For example, the free iron can be provided at a final concentration as low as between about 0.1 μM and about 100 mM free iron. Optionally, the concentration of free iron is between about 0.1 μM and about 1 mM free iron, preferably about 1 μM free iron. The species (or groups of species) to be reduced in population play a role in determining the treatment parameters; the length of time sufficient to decrease the microbial population can be determined using methods for evaluating microbiocidal activity known to one of skill in the art. This sufficient length of time is easily determined by one practicing the methods of the present invention.

The free iron and the stress inducer can be coadministered, or they can be applied to the surface sequentially. Optionally, the free iron can be applied first, and subsequently removed prior to bringing the surface into contact with the stress inducer. In this embodiment, for example, approximately 50%, 75%, 90%, 95%, 98%, 99%, 99.5%, or substantially all of the free iron is optionally removed. Removal of the free iron can be achieved by various methods known to one of skill in the art.

Treatment of animal surfaces

The methods of the present invention can also be used to reduce the population of an animal surface, for example, a poultry intestinal tract, prior to sacrificing the animal and processing of the carcass. Meat products processed in assembly-line processing plants (we use poultry as an example, but the methods contemplate any animal carcass) represent an important source of human exposure to bacterial pathogens, and methods for reducing bacterial load on processed poultry products has received considerable attention. Both the interior and exterior surfaces of eviscerated poultry carcasses carry a burden of bacterial cells, due to contamination of birds both by their own feces and by other resident organisms prior to their arrival in the processing plant, as well as due to contamination caused by the unavoidable spread of bacteria during removal of the birds' digestive tracts. A standard method for the disinfection of residual bacterial contamination involves passage of carcasses through a water chiller into which hypochlorite (bleach) is used as both a disinfectant and as a means of preventing cross-contamination. Although chlorination has been shown to be reasonably effective, there are distinct disadvantages to its use, including dangers posed to consumers, workers and the environment, and concerns about its effect on the quality of chickens so treated. For example, exposure of meat to chlorine results in the formation of organochlorine compounds that are known to be mutagenic and carcinogenic. Also, the handling of hypochlorite in large quantities is hazardous, due to the possibility of chemical spills and the potential for the generation of chlorine gas. Moreover, hypochlorite must be inactivated before the treatment water from the water chiller may be returned to the environment, as chlorine is highly toxic. Lastly, there are concerns surrounding the undesirable taste and odor of chlorine residues in the treated chickens.

The above example of chlorine use in poultry processing illustrates a central problem with many common and widely used disinfectants, namely, that they are harsh reagents in general, the effectiveness of which derives from their broad destructiveness. In other words, many antimicrobial treatments are sufficiently corrosive, caustic, denaturing, oxidizing, emulsifying, or otherwise harsh that they are equally if not more toxic to eukaryotic cells (such as human cells) than they are to microbes, and are therefore correctly categorized as biocidal, i.e., compounds which kill cells of all life forms, microbial and non-microbial.

The present invention provides methods for reducing a microbial population on an animal surface. The methods include the steps of a) providing a composition of free iron; b) providing a non-oxidant stress inducer; and c) bringing the animal surface into contact with the free iron and the non-oxidant stress inducer, thereby reducing the microbial population on the food surface. Any number of transition metals can be employed in the methods of the present invention, but a particularly preferred transition metal is iron. The transition metal (or combination of transition metals) is provided, for example, as a salt, an organic complex, or a combination thereof. Exemplary salts and/or organic complexes include, but are not limited to, acetate, ammonium citrate, ammonium oxalate, ammonium sulfate, bromide, chloride, citrate, fluoride, fumarate, hydroxide, iodide, nitrate, oxide, phosphate, pyrophosphate, sulfate, and tartrate. The metal salt and/or organic complex can be prepared in water, or it can be prepared in a formulation (e.g., a buffer) suitable for the intended use. Optionally, the iron formulation is prepared in water or a hypo-osmotic saline solution.

The methods of the present invention also provide one or more oxidant or non-oxidant stress inducers. Preferred stress inducers for use in the methods of decontaminating a animal surface include, but are not limited to, the non-oxidant stress inducer lysozyme, and physical disinfectants (e.g., osmotic shock). However, other forms of stress inducers can be employed in the methods for treating an animal surface.

The animal surface to be treated in the methods of the present invention include, but are not limited to, an outer body surface, a mucosal, surface, a digestive tract, or a combination thereof. Contacting the animal surface with the free iron and stress inducer can be accomplished by any of a number of techniques as described previously, including spraying, dipping, or feeding the free iron and stress inducer composition to the animal. Furthermore, the surfaces can be treated, for example, either prior to sacrificing the animal, or afterwards.

In one preferred embodiment of the methods of the present invention, bringing the animal surface into contact with the free iron and the non-oxidant stress inducer includes feeding the animal a composition of free iron prior to sacrifice, or slaughter, of the animals. The iron is applied “enterically” (e.g., it is incorporated into their feed) to eliminate or reduce the number of viable microbial organisms in the digestive tract of an animal destined for slaughter. In this embodiment, animals are fed an iron-rich diet prior to slaughter in such a way that a transient increase in iron concentration occurs throughout the digestive tract, such that bacteria which reside within the digestive tract are exposed to a lethal combination of the metal ions. In one embodiment of this method, the animals are fed a stress inducer (optionally, a non-oxidant stress inducer) concomitant with, or subsequent to, contacting the animal surface (i.e., the digestive tract) with the free iron preparation; in other embodiment, the stress inducer is contacted with the animal surface after sacrifice of the animal. It is a novel and useful aspect of the invention that iron concentrations are titrated such that the microbes (but not the animals) are adversely affected. It is disclosed that iron concentrations can be antimicrobial per se, but alternatively or additionally, may be antimicrobial in that enteric microbes, having been exposed to iron within the digestive tract of animals, are thereby significantly sensitized to treatment with a stress inducer (optionally, a non-oxidative stress inducer) upon later exposure during the processing of the carcasses.

Furthermore, a range of concentrations of free iron can be employed in the methods for reducing a microbial population on a animal surface. For example, the free iron can be provided at a concentration as low as between about 0.1 nM and about 1 M free iron. Optionally, the concentration of free iron is between about 0.1 μM and about 100 mM free iron, preferably, 0.1 μM and about 1 mM free iron, and more preferably about 1 mM free iron. The animal surface to be treated is exposed to the free iron and/or stress inducer for a length of time sufficient to decrease the microbial population. This sufficient length of time is easily determined by one practicing the methods of the present invention. For example, the animal surface can be exposed for as short a time period as 5 seconds, or the amount of time that it would take to dip the animal article into a preparation of the free iron. Alternatively, the free iron can be applied to the animal surface and left there (i.e., not specifically removed or washed away after treatment of the surface). Times of exposure between these two extremes are also contemplated in the methods of the present invention, as described in greater detail in previous sections. The free iron and the stress inducer can be coadministered (e.g., if the animal is to be fed the preparation prior to being sacrificed), or they can be applied to the surface sequentially. Optionally, the free iron can be applied first, and subsequently removed prior to bringing the surface into contact with the stress inducer. In this embodiment, for example, approximately 50%, 75%, 90%, 95%, 98%, 99%, 99.5%, or substantially all of the free iron is optionally removed. Removal can be performed, for example, by allowing the iron preparation to pass partially or completely through the digestive tract of the animal, prior to application of the stress inducer. The optimum concentrations and exposure durations can be determined empirically for the animal surface to be treated (e.g., taking into account the type of animal surface, and extent of exposure the animal surface can withstand). Furthermore, the species (or groups of species) to be reduced in population can play a role in determining the treatment parameters, using methods for evaluating microbiocidal activity known to one of skill in the art.

Additional Components And Methods

While implementing any of the methods of the present invention, further components are optionally provided, such as one or more siderophores and/or biocide enhancers.

Biocide Enhancers

The methods of the present invention can further include the step of providing one or more biocide enhancers. Biocide enhancers act in a manner similar to, and have as a subcategory, the stress inducers described previously. Exemplary biocide enhancers include, but are not limited to, riboflavin, flavenoids, photo-activatable compounds, phenols, cetyl pyridinium chloride, trisodium phosphate, hydrogen peroxide, bleach, one or more fatty acids, one or more organic acids, citric acid, and ascorbic acid. These components act in concert with the free iron and stress inducer to further decrease the microbial population present on a surface to be treated. For example, addition of flavin compounds (e.g. riboflavin) in combination with intense white light lead to photosensitization of the microbes, enhancing the effectiveness of the transition metal/stress inducer treatment.

Biocide enhancers that interfere with the binding of microbial organisms to the surface to be treated are also considered in the present invention. Binding inhibitors optionally employed in the methods and compositions of the present invention can, for example, compete with a microbial population for receptors on a cell surface. Exemplary biocide enhancers that function as binding inhibitors include, but are not limited to, lectins and polysaccharides (such as those described in U.S. Pat. Nos. 6,126,961 to Kross; 5,998,381 to Shekhani et al.; 5,902,796 to Shand et al.; and 5,703,060 to McAnalley et al.).

Siderophores

The methods of the present invention further include providing one or more siderophores during the population reduction treatment. Siderophores are iron-binding factors utilized (and often produced) by microbes (both prokaryotic and eukaryotic) for sequestering iron (generally, ferric iron, or Fe³⁺) from the environment. Generally, the siderophore structures are low-molecular weight moieties having a high-affinity for iron. Most commonly, siderophores are used by the microbe, in an attempt to compete with other cells (often a host organism, in the case of invasive microbial infection) for limited quantities of iron. Generally, environmental iron is not available as free iron, but rather is bound to various chelators or proteins, such as hemoglobin, transferrin, and/or lactoferrin. Since iron is required for cell viability, one way in which microbes can compete for the limited quantities of iron typically available in the environments is by the synthesis and us of the siderophore molecules. Since various microbes are capable of producing siderophores, the antimicrobial effects of providing a source of free iron and one or more siderophores are a surprising feature of the present invention.

Siderophores are produced by a number of bacteria, fungi, and some plants. Exemplary microbes which produce siderophores include, but are not limited to, Aeromonas, Alcagenes, Escherichia coli, Erwinia, Frankia, Pseudomonas, Ralstonia, Rhizobium, Salmonella, and Vibrio. The synthesis of siderophores involves multiple microbial gene products, and the resulting siderophore structure can only be used once; typically, the molecule must be cleaved in order to free the iron for cellular use. Thus, the synthesis and use of siderophores is a very energy-expensive process for the microbial cell.

The three structural classifications of siderophores include hydroxamate-based structures, catechol-based structures, and phenolate structures. The siderophores have a very high binding constant (on the order of around 10⁴⁹ to 10⁵³) and thus are able to efficiently compete for iron or other transition metals. Additionally, many of the siderophores can form complexes with other metal ions, such as gallenium Ga(III), chromium Cr(IIE) and vanadium V(IV). Once the iron is bound, the siderophore is then recognized by cell surface receptors and taken into the cell.

The present invention provides siderophore-based methods for reducing a microbial population on a surface. The methods include the steps of a) providing a preparation of free iron; b) providing one or more siderophores; and c) bringing the surface into contact with the free iron and the one or more siderophores, thereby reducing the microbial population on the surface. The preparation of free iron (or combination of iron and other transition metals) is provided, for example, as a salt, an organic complex, or a combination thereof. Exemplary salts and/or organic complexes include, but are not limited to, acetate, ammonium citrate, ammonium oxalate, ammonium sulfate, bromide, chloride, citrate, fluoride, fumarate, hydroxide, iodide, nitrate, oxide, phosphate, pyrophosphate, sulfate, and tartrate. The metal salt and/or organic complex can be prepared in water, or it can be prepared in a formulation (e.g., a buffer) suitable for the intended use. Optionally, the iron formulation is prepared in water or a hypo-osmotic saline solution.

The one or more siderophores include, but are not limited to, aerobactin, alcaligin, cepabactin, desferriferrichrysin, desferriferricrocin, desferriferrioxamine B, desferriferrioxamine E, coprogen, corrugatin (a lipopeptide siderophore), enterobactin, enterochelin, exochelin, ferrichrome, ferrioxamine, gallichrome, mycobaction, myxochelin, nocardamine, pseudobactin M114, pyoverdine, pyochelin, pseudobactin St3, rhizoferrin, rhodotorulic acid, schizokinen, pseudobactin 7NSK2, trencam, WCS, and vibriobactin. Enterobactin, for example, is a cyclic triester of 2,3-dihydroxy-N-benzoyl-L-serine. Aerobactin is a conjugate of 6-(N-acetyl-N-hydroxylamine)-2-aminohexanoic acid and citric acid. Additionally, compounds such as salicylic acid can also function as siderophores.

The siderophore-based methods of the present invention can be used to reduce the microbial population on a variety of surfaces, such as a food surface, an animal surface (e.g., an outer body surface, a digestive tract, or a combination thereof), an environmental surface, a piece of medical equipment, a wound, an abrasion, a burn, or a damaged region of tissue. Contacting the surface with the free iron and siderophore can be accomplished by any of a number of techniques as described previously for methods involving free iron and a stress inducer. The free iron preparation can be applied separately from the siderophore, the two compositions can be coadministered, or the two compositions can be combined prior to application to the surface. For example, the surface can be sprayed with the low concentration of free iron and the siderophore. Alternatively, the surface can be either completely or partially submerged, or “dipped,” into the free iron and/or siderophore preparations. The antimicrobial preparations can be applied with a sponge, a mop, a cloth, or any other of a variety of techniques known to one of skill in the art of disinfection. Furthermore, combinations of these application techniques can be employed (for example, the surface may be submerged in the free iron preparation, and subsequently sprayed with the stress inducer preparation).

Optionally, the surface is exposed to one or more of the free iron preparation and/ siderophore for a transient length of time suitable to reduce the microbial population on the surface. Suitable lengths of time will depend, in part, upon the surface to be treated, as well as the microbial population to be reduced. For example, a suitable length of time for exposure of the surface to one or more of the free iron and the siderophore range from 30 seconds to one week. Exemplary lengths of time include, about 30 seconds, about 3 minutes, about 1 hour, about 4 hours, about 12 hours, about 24 hours, and about 1 week.

Furthermore, a range of concentrations of free iron can be employed in the methods for reducing a microbial population on a food surface. For example, the free iron can be provided at a concentration as low as between about 0.1 nM and about 1 M free iron. Optionally, the concentration of free iron is between about 0.1 μM and about 100 mM free iron, preferably, 0.1 μM and about 1 mM free iron, and more preferably about 1 μM free iron. The surface to be treated is exposed to the free iron and siderophore for a length of time sufficient to decrease the microbial population. This sufficient length of time is easily determined by one practicing the methods of the present invention. For example, a food surface can be exposed for as short a time period as 5 seconds, or the amount of time that it would take to dip the food article into a preparation of the free iron. Alternatively, the free iron can be applied to the surface of a medical instrument or a countertop, and left there (i.e., not specifically removed or washed away after treatment of the surface). Times of exposure between these two extremes are also contemplated in the methods of the present invention, as described in greater detail in previous sections. Optionally, the free iron and the siderophore are coadministered. The optimum concentrations and exposure durations can be determined empirically for the surface to be treated (e.g., taking into account the type of surface, physical characteristics of the surface (e.g., porosity), and extent of exposure the surface can withstand). Furthermore, the species (or groups of species) to be reduced in population can play a role in determining the treatment parameters, using methods for evaluating microbiocidal activity known to one of skill in the art.

Optionally, the methods of the present invention further include with one or more oxidant or non-oxidant stress inducers. Preferred stress inducers for use in the methods of decreasing the microbial population on a surface of include, but are not limited to, halides (e.g. iodine and iodophores, bromine, chlorine and chlorine-based products), organic solvents (e.g. ethanol), organic and inorganic acids (e.g. lactic acid, acetic acid, hydrochloric acid, nitric acid), bases (e.g. trisodium phosphate, quaternary amines), and aldehydes (formaldehyde, glutaraldehyde). Additional chemical disinfectants include, but are not limited to, lytic enzymes (e.g. lysozyme), oxidants (e.g. peroxides, hypochlorite, ozone), sorbates, carbohydrate polymers (e.g., chitin, chitosan, β(1→) acetylmannans), fatty acids and oils, as well as soaps, detergents and other surfactants. The optional stress inducer can be coadministered, or they can be applied to the surface sequentially. Optionally, the free iron and siderophore can be applied first, and subsequently removed prior to bringing the surface into contact with the stress inducer. In this embodiment, for example, approximately 50%, 75%, 90%, 95%, 98%, 99%, 99.5%, or substantially all of the free iron and/or siderophore is optionally removed.

Adhesion and/or Thickening Agents

The methods and compositions of the present invention can optionally further comprise a thickening agent, or an adhesive agent, to enhance the contacting of the surface with the free iron preparation. Optionally, polysaccharides are employed to enhance the contacting of the surface. Examples of polysaccharides which can optionally be included in the methods and compositions of the present invention include, but are not limited to, acacia, agar, carrageenan, chitin, chitosan, chondroitin, chondroitin sulfate, cellulose and cellulose derivatives (e.g., hydroxyethyl cellulose, hydroxypropyl cellulose, methylhydroxypropyl cellulose, methyl cellulose, carboxymethyl cellulose), curdlan, dermatan sulfate, dextran, dextran sulfate, galactan, glycogen, guar gum and derivatives (e.g., hydroxyethyl guar gum, carboxymethyl guar gum), heparin and heparin derivatives (e.g., low molecular weight heparins, modified heparins), heparan sulfate, hyaluronic acid, sodium hyaluronate, keratan sulfate, locust bean gum, mannan, mucopolysaccharides, pectin, quinsseed, starch, succinoglucane, tragacanth gum, xyloglucan, and xanthan gum. Additional agents can be found, for example, in U.S. Pat. Nos. 6,197,318 to Abe, et al. and 5,972,857 to Roselle, et al.

Furthermore, the compositions of the present invention can optionally include one or more of chemical foaming agents (e.g., saponin) or adjuvants, to increase access of solutions to contaminating microbes on the surfaces of food items, adherence of compositions to food items for the purpose of increasing treatment duration, and so forth. Preferably, these agents do not precipitate the redox metals, do not interfere with the uptake of the metals by microbial cells, and do not interfere with the action of the stress inducers. For example, since alkaline pH causes oxidation and precipitation of iron ions, basic solutions are less preferable than acidic solutions. Similar considerations will be obvious to those skilled in the art of preparing solutions containing transition metals such as iron or copper.

### EXAMPLES

Classical methods for determining antimicrobial effects involve exposure of cells to disinfectant or antiseptic agents contained in tubes or vials, removal of the agents by centrifugation, washing of the cells to remove disinfectant residues, plating of the cells at a number of dilutions onto culture plates, and enumeration of the resulting colonies. Such methods are inadequate for the purposes of the present invention for two principle reasons, namely, treatment of cells in tubes is too lengthy for the determination of rapid effects (e.g., on the order of 1-5 minutes), and treatment and plating are too laborious for the accurate determination of many different interactions in parallel. For the purposes of this invention, the classical plate-based methods have been replaced by a high-throughput liquid outgrowth system described below. In these experiments, the assays were prepared as follows, and quadruplicate wells were treated in parallel.

Exemplary preparations of free iron and various oxidant and/or non-oxidant stress inducers are described in greater detail below.

Methodology

A high-throughput 96-well liquid outgrowth system was devised for the measurement of bacterial cell killing. Known numbers of living cells were treated, and unknown numbers of surviving cells were quantified by measuring the rate of liquid outgrowth of the culture relative to a standard curve, as determined by optical density.

Cells of Escherichia coli (ATCC strain #25404) were grown to mid-log cell density (absorbance of about 0.1 at 600 nm) in 100 mL of Luria Broth (LB) at 37° C. in Erlenmeyer flasks, and then chilled at 0° C. on ice prior to use. “Treatment” plates were prepared as follows. Chilled cells were aliquoted (approximately 50 μL) into wells of a 96-well filtration plate (Multiscreen® (Millipore Corporation, Bedford, Mass.), having a 0.22 μM hydrophilic Durapore® membrane at the bottom of the wells). The LB medium was removed by vacuum filtration, and the remaining cells were washed with 150 μL of an iso-osmotic solution, either sterile saline solution (SSS, 130mM NaCl, pH 5.0) or sterile HEPES-buffered saline (HBS, 0.595% HEPES, 0.82% NaCl, pH 7.05).

The cells on the treatment plate were then exposed to various free iron (for example, FeCl₂) and optional stress inducer treatments as described in the examples delineated below. The FeCl₂ solutions were always prepared fresh on the day of use from the anhydrous salt. The cells in a set number of wells were not treated; these untreated cells were serially diluted and used to construct a standard curve.

“Reading” plates were made from the treatment plate by adding approximately 130 μL of sterile LB medium to each well of three sterile polycarbonate microtiter plates (Corning). Fifty micrometers of the resuspended cells from all wells of the treatment plate were then transferred to a corresponding well on the reading plates, which were then sealed (for example, using polypropylene sealing mats) and incubated at 37° C. with vigorous shaking. Absorbance readings of turbidity (measured atλ=600 nm) were recorded at approximately 45minute intervals over a time course ranging from about two to about seven hours. Cell densities of the wells containing the treated cells were calculated by comparison to the standard curve.

Absorbance readings were performed at 600 nm using a standard microtiter plate absorbance reader (Multiskan® microplate reader by Labsystems, Helsinki, Fla.). The measurements collected for the treated cells were disregarded unless they fell within a region of linear growth in liquid culture, which was individually determined for each time point reading. Generally, acceptable measurements ranged from about 0.020 to about 0.200 OD units. Cell densities for treated cells were calculated by comparison of absorbance readings to the standard curve (typically representing a range from about 0.0013% to about 20% of the original viable cell density). Techniques for calculating slopes and determining concentrations from standard curves are known to one skilled in the art,

### EXAMPLES 1

### Antimicrobial Effects Of Iron And Hydrogen Peroxide.

FeCl₂ solutions were prepared from the anhydrous salt, and peroxide (H₂O₂) dilutions are prepared fresh from a 30% w/w stock solution. Cells were plated as described above, then treated with FeCl₂ at concentrations ranging from about 1 μM to about 1.024 mM (in HBS). Cells were exposed to the FeCI₂ solution for approximately 10 minutes at room temperature (i.e., iron “pre-treatment”). The control wells and standard wells were treated with HBS alone (no FeCl₂). Cells were subsequently exposed to hydrogen peroxide at concentrations ranging from about 73 μM to about 1 mM (in HBS) for an additional 10 minutes at room temperature (controls and standards were again treated with HBS alone). The cells were then washed by passing approximately 2 ml of HBS (in 250 μL increments) through each well.

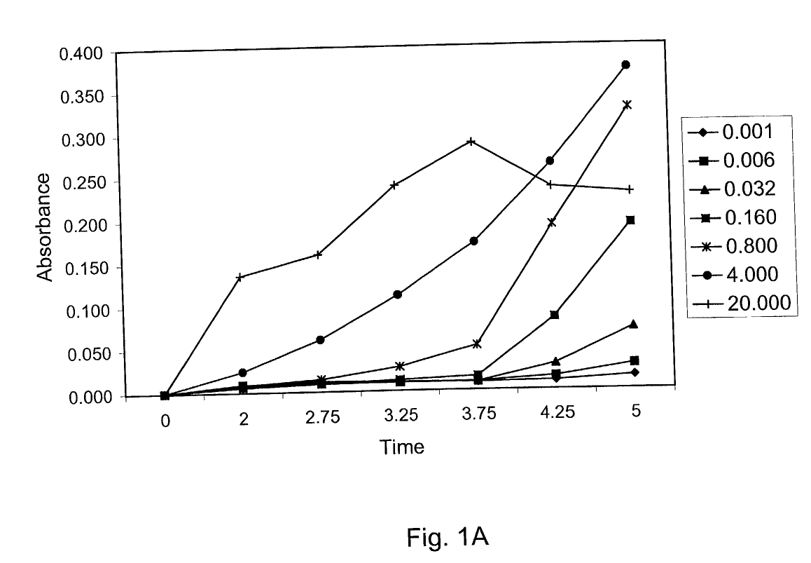

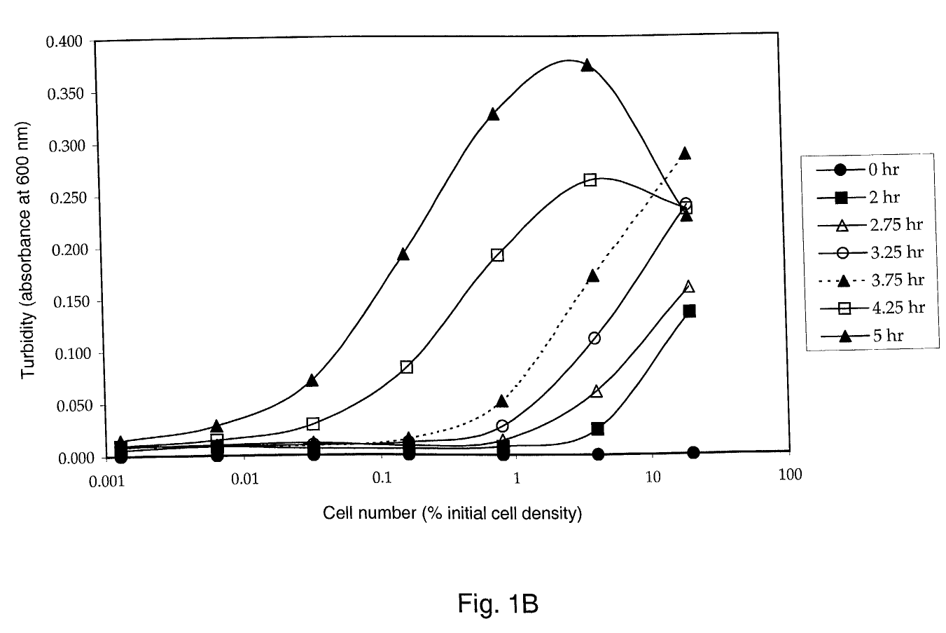

FIG. 1B shows the absorbance readings at 600 nm of a number of E. coli cell cultures started at different initial cell densities (0.001, 0.006, 0.032, 0.16, 0.8, 4, and 20% of an initial starting culture) as recorded at a number of time points between 0 and 5 hours. At lower ranges of initial cell densities, the linear portion of the standard curve falls over increasingly later time points. The standard curve was determined to provide accurate cell densities across a range of concentrations, having as its upper limit a number of cells equivalent to approximately 20% of the initial number of treated cells, and as its lower limit a number of cells equivalent to about 0.0013% of the original viable cell density. Thus, in these experiments, “quantifiable” cell killing was limited to that resulting in ≧0.7 log reduction and ≦4.9 log reduction in viable cell number.

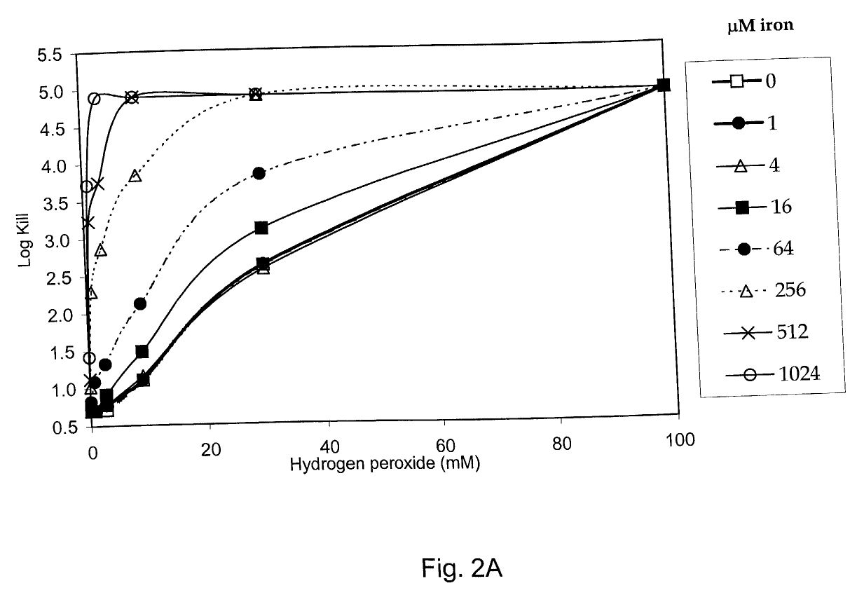

FIG. 2, panels A and B, illustrate the decreases in cell viability (i.e., cell killing) resulting from treatment of E. coli with FeCl₂ and hydrogen peroxide. As is shown in FIG. 2 panel A, FeCl₂ treatment, in the absence of hydrogen peroxide as a stress inducer, did not result in appreciable cell killing at any concentration. In contrast, treatment of the cells with the free iron solution prior to exposure to H₂O₂, at concentrations as low as 731μM H₂O₂, reduced the microbial populations significantly. At higher concentrations of H₂O₂ even more dramatic effects of iron pre-loading were seen. The observed saturation of the effect at a kill of 4.9 log is merely a reflection of the detection range limitation on this assay imposed by the lower limit of the standard curve.

FIG. 2, panel B, which depicts the same data as panel A but with the axes reversed, illustrates that the FeCl₂ “pre-treatment” sensitized the cells to subsequent exposure to the stress inducer H₂O₂. At concentrations of 16 μM and higher, FeCl₂ pre-treatment increases the sensitivity of cells to subsequent H₂O₂, decreasing the concentration of H₂O₂ required to achieve a given degree of killing.

### EXAMPLES 2

### Antimicrobial effects of iron, H₂O₂, and hypo-osmotic shock.

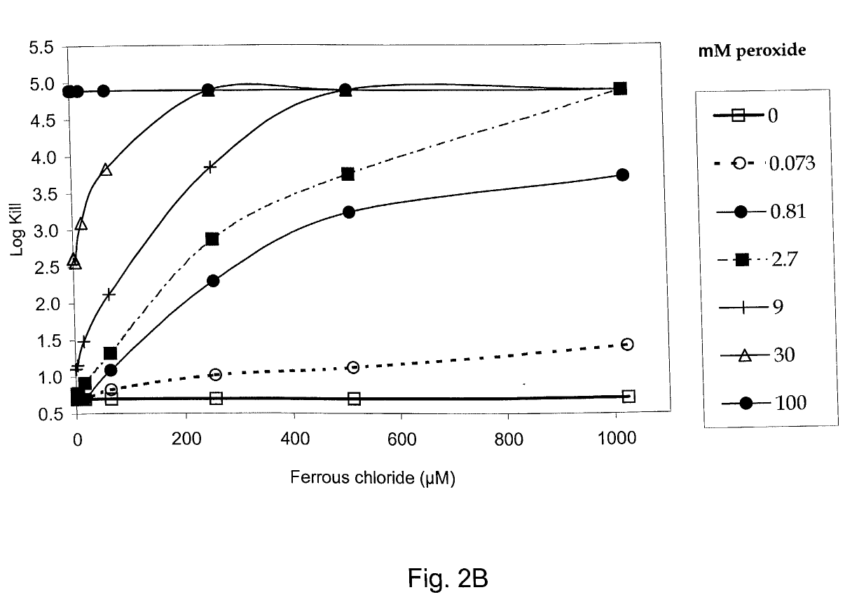

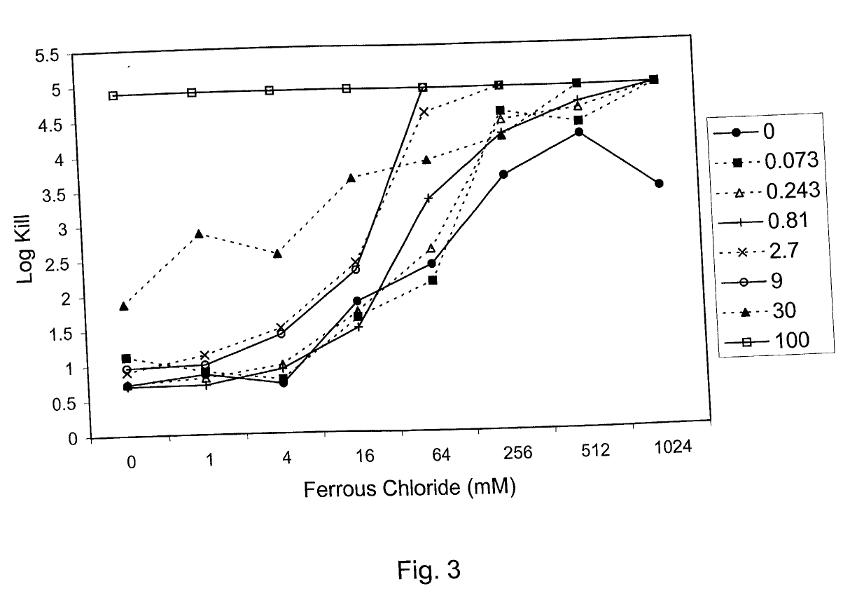

Cells were prepared as described previously, then treated with FeCl₂ at concentrations ranging from about 1 μM to about 1.024 mM (in H₂O at approx. pH 5, instead of KBS). Cells were exposed to the hypo-osmotic FeCl₂ solution for approximately 10 minutes at room temperature. The cells were subsequently exposed to hydrogen peroxide at concentrations ranging from about 73 μM to about 1 mM (also in H₂O at approx. pH 5) for an additional 10 minutes at room temperature. The cells were then washed by passing approximately 2 ml of BBS (in 250 μL increments) through each well. FIG. 3 depicts the effects that free iron and peroxide had on cell killing rates in a hypo-osmotic environment, as compared to the iso-osmotic environment of Example 1. The pH of the solutions in the two experiments (with and without what) was equivalent (pH 5), hence the experimental parameters differed only in osmotic strength. It was surprisingly noted that even in the absence of the peroxide treatment, exposure of the cells solely to the FeCl₂ solution had a substantial antimicrobial effect. FeCl₂ concentrations between about 256 μM and about 1.024 mM resulted in a log kill of between 0.7 and 4.9. It was previously shown in

FIG. 2, panel A, shows that iso-osmotic FeCl₂ in the absence of the stress inducer peroxide had a relatively small antimicrobial effect on the microbial populations. The antimicrobial effect of free iron was enhanced by a non-oxidant stress inducer, in this case, the physical stress resulting from hypo-osmotic shock.

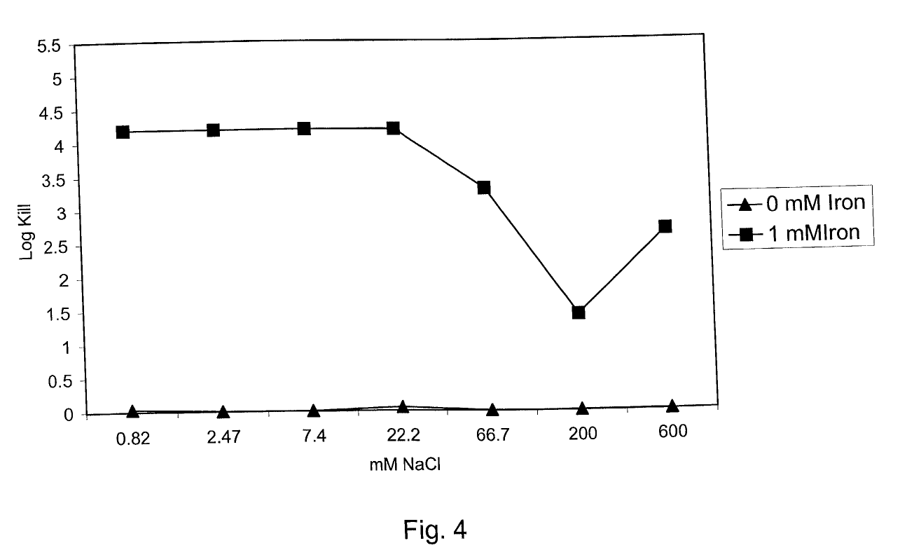

Furthermore, cells were treated for 10 min with or without 1 mM FeCI₂ in iso-osmotic saline prior to treatment for 10 min in a range of osmolarities of sodium chloride (FIG. 4). Saline treatment alone does not have an antimicrobial effect, at any of the NaCl concentrations tested; however, treatment with free iron resulted in killing at all osmolarities. In these experiments, free iron (alone) was minimally effective near the iso-osmotic point (130 mM) and maximally effective as an antimicrobial treatment under hypo-osmotic (e.g., 0 mM NaCl) or hyper-osmotic (600 mM) conditions. In other words, hypo-osmotic shock in combination with exposure to free iron is deleterious to cell viability.

### EXAMPLES 3

### Antimicrobial Effects Of Iron And Cetyl Pyridinium Chloride

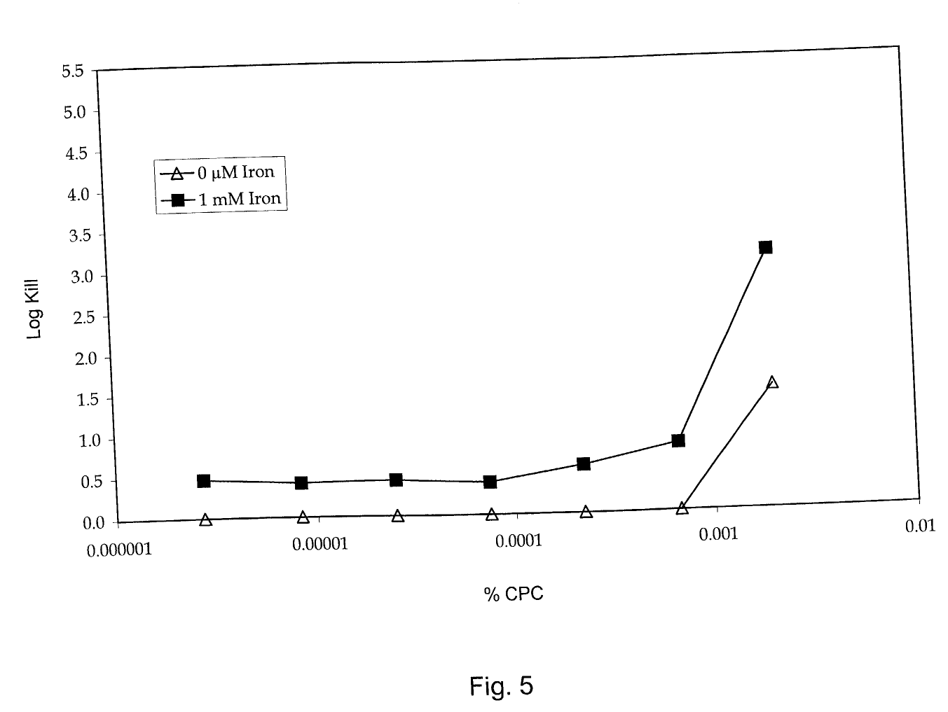

Similar experiments were performed using 1 mM FeCl₂ in iso-osmotic saline (130 mM NaCl, pH 5.0) and a range of concentrations of cetyl pyridinium chloride (CPC). FIG. 5 depicts the log kill due to cellular exposure to free iron and CPC as the stress inducer; the antimicrobial effect of CPC was potentiated by pre-incubation with iron.

### EXAMPLES 4

### Antimicrobial Effects Of Iron And Citric Acid

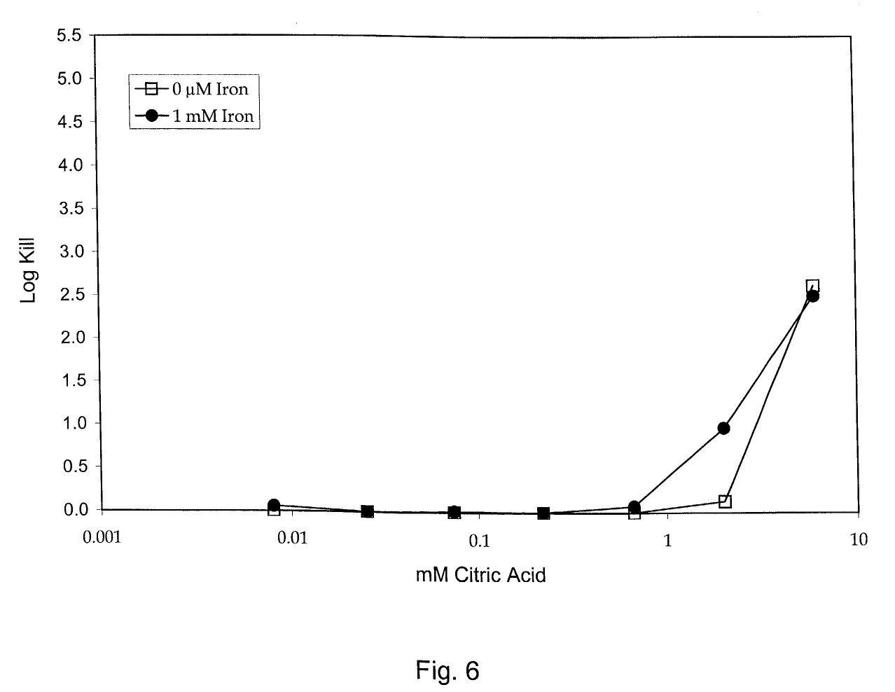

Cells were treated for 10 minutes with or without 1 mM FeCl₂ in iso-osmotic saline (130 mM NaCl at pH 5.0) prior to treatment for 10 minutes with a range concentrations of citric acid. As is evident in FIG. 6, the log kill due to citric acid was potentiated by pre-incubation with iron.

### EXAMPLES 5

### Antimicrobial Effects Of Iron And Hypochlorite.

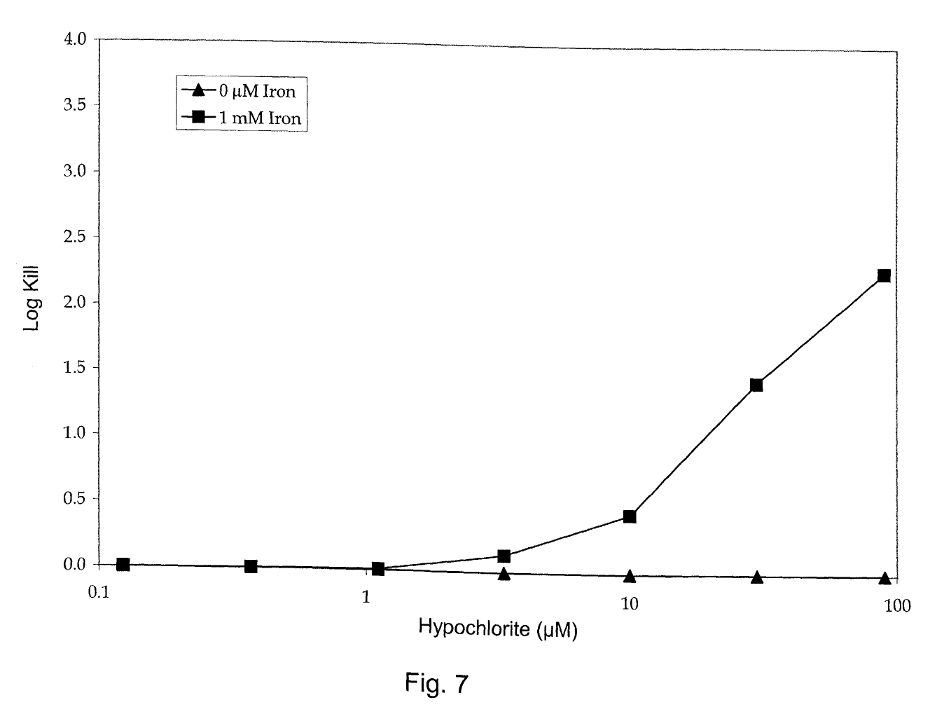

Similar experiments were performed using 1 mM FeCl₂ in iso-osmotic saline (130 mM NaCl, pH 5.0) and a range of concentrations of the oxidant hypochlorite (e.g., bleach). As depicted in FIG. 7, none of the concentrations of bleach used resulted in any kill alone. However, these concentrations were highly effective when administered to cells previously exposed to free iron.

### EXAMPLES 6

### Antimicrobial Effects Of Iron And Lysozyme.

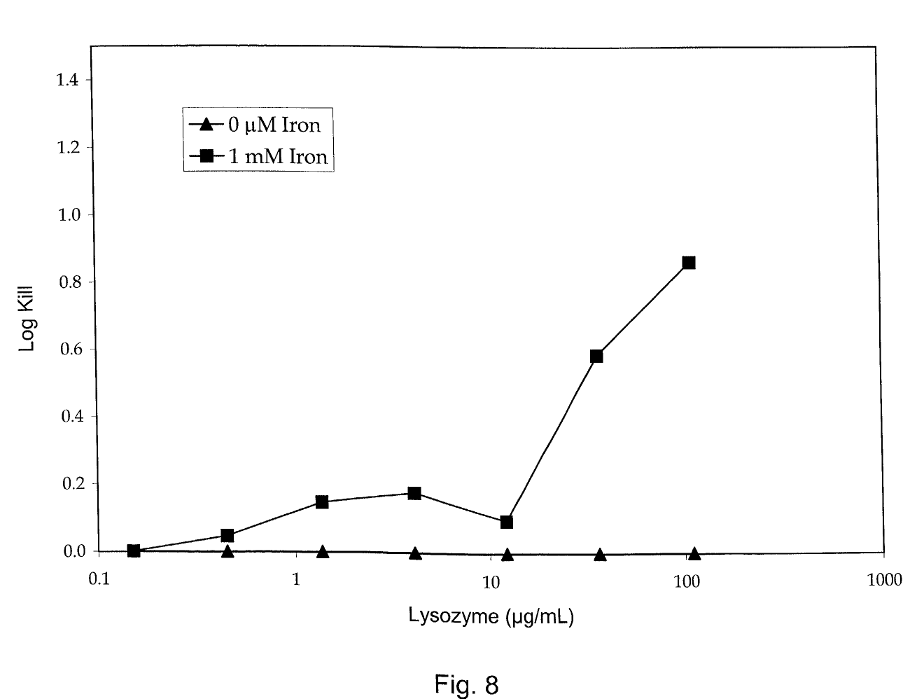

Cells were treated for 10 min with 1 mM FeCl₂ in iso-osmotic saline, prior to treatment for 10 min with a range concentrations of the proteolytic enzyme lysozyme. As shown in FIG. 8, whereas none of the concentrations of lysozyme used resulted in any kill alone, these concentrations were highly effective when administered to iron-sensitized cells.

### EXAMPLES 7

### High-Throughput Determination Of Antimicrobial Killing Iron And Trisodium Phosphate.

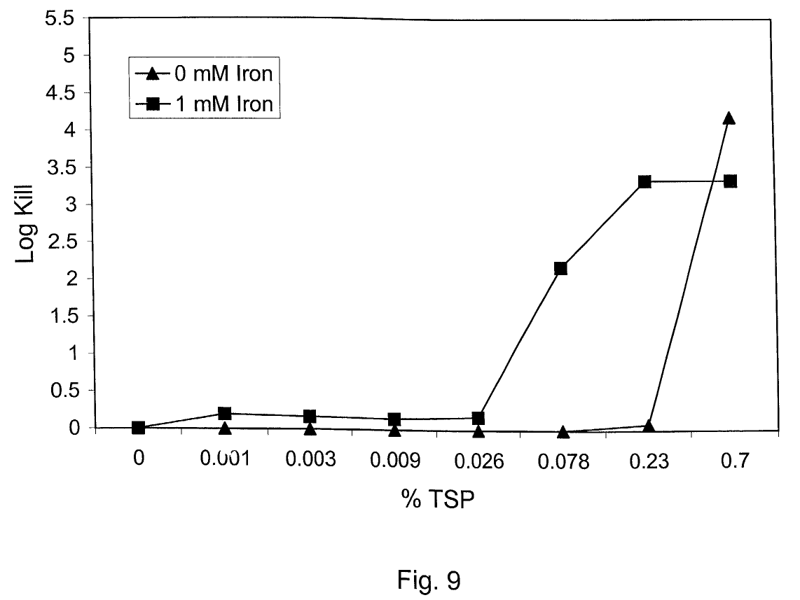

Similar experiments were performed using 1 mM FeCl₂ in iso-osmotic saline and a range of concentrations of trisodium phosphate (TSP). As depicted in FIG. 9, the antimicrobial effects of TSP were enhanced by pre-incubation of the cells with free iron.

Compositions For Reducing Microbial Populations

The present invention also provides novel compositions for reducing a microbial population on a surface. The compositions include a preparation of free iron, as described in the previous sections. Optionally, the compositions further include one or more non-oxidant stress inducers, one or more iron-binding compounds (such as chelators and/or siderophores), one or more polymers (e.g., chitin, chitosan, other polysaccharides) or combinations thereof.

Wound Dressings

In one embodiment, the compositions of the present invention include a preparation of free iron and a wound dressing component. Wound dressing components are generally composed of one or more absorbent or bibulous materials, and as such are known to one skilled in the art. Absorbent materials which can be employed in the compositions of the present invention include, but are not limited to, gauze, cotton, cotton fibers, natural or synthetic sponges, plastic fibers and fabrics, and the like. Wound dressing components also include suture materials, such as biodegradable sutures. Exemplary biodegradable components which can be employed in the compositions of the present invention include, but are not limited to, polylactides, polyglycolides, polycaprolactones, polyamino acids, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), poly(methyl vinyl ether), poly(maleic anhydride, or their copolymers.

The wound dressing composition of the present invention optionally further comprises a stress inducer, preferably a non-oxidant stress inducers. Any of the stress inducers (both oxidant and non-oxidant) described in previous sections can be employed in the wound dressing compositions of the present invention. One preferred non-oxidant stress inducer is chitosan (or chitin, or a derivative thereof). Another preferred non-oxidant stress inducer is aloe, including the component carbohydrate polymer β(1→4) acetylated mannan, as well as aloe derivatives (such as acemannan).

Additional materials which may be used as a wound dressing component in the composition of the present invention include, but are not limited to, starch and starch derivatives; methyl cellulose, carboxymethyl cellulose, hydroxypropyl cellulose and other cellulose derivatives; natural gums, such as alginates, xanthan gum, locust bean gum; alkali metal and ammonium salts of poly(acrylic acid) and poly(methacrylic acid); polyacrylamides; polyolefins; polyvinylethers; polyvinylpyrrolidone polyvinylmorpholinone; polyvinylalcohol; and mixtures and copolymers thereof. See, for example, U.S. Pat. Nos. 6,177,607 (Blaney, et al.) and 6,190,768 (Turley, et al.).

Optionally, the wound dressing component of the present invention includes an adhesive element, such as a pressure-sensitive adhesive (PSA). Exemplary adhesive elements include, but are not limited to, polyisobutylenes, silicone-based adhesives, acrylate adhesives, and the like. Furthermore, the wound dressing component optionally further comprises a backing material (typically placed against the adhesive side of the dressing). Optionally, the backing material is an occlusive material (i.e., a material that is prevents fluid passage). The backing materials described in PCTIUS90/04767, the disclosure of which is incorporated herein by reference, may be used in the devices of this invention.

Additional components that enhance the process of wound healing can be included in the composition of the present invention, including, but not limited to, the compositions described in U.S. Pat. No. 6,187,743 (Obi-Tabot), which publication is incorporated herein in its entirety.

Lotions and Lubricants

In an alternative embodiment of the present invention, the composition includes a preparation of free iron and a lubricant or lotion. The lotion compositions of the present invention can optionally include a non-oxidant stress inducer, such as those listed previously. The lubricant or lotion can include various components common to the pharmaceutical and/or cosmetics industry, such as algael extracts, allantoin, aloe vera, alpha hydroxy acids (e.g., glycolic acid, lactic acid) and beta hydroxy acids (e.g. salicylic acid), amino acids, ammonium lauryl sulfate, ascorbic acid (vitamin C), benzoyl peroxide, bioflavinoids, ceramides, clays (including, but not limited to, bentonite and kaolin), cocoa butter, collagen, corn starch, cyclomethicone, dimethicone, elastin, fatty acids (e.g., linoleic, oleic), various glycols (including, but not limited to butylene glycol, hexylene glycol, propylene glycol, polyethylene glycol, and the like), glycerin, glycerhizinate compounds (such as ammonium glycerhizinate), glutathione, glycoaminoglycans, glycosphingolipids, hyaluronic acid, hydroquinones, lanolin and lanolin derivatives, lecithin and other lectins, licorice root, liposomes, magnesium lauryl sulfate, various oils (including, but not limited to, apricot kernel, avocado, castor, clove, coconut, corn, cottonseed, eucalyptus, fennel, grapefruit, jojoba, lavender, lemon, lemongrass, lime, palm, rose, soybean, sunflower seed, wheat germ, and the like), parabens (e.g., methyl, propyl), mineral oil, polysaccharides and/or mucopolysaccharides, phospholipids, retinol (vitamin A), salicylic acid, silicone oil (and other mineral oils), sodium lauryl sulfate, squalene, tocopherol (vitamin E), triglycerides (including, but not limited to, caprylic, capric, lauric), and urea and urea derivatives.

Furthermore, anti-inflammatory components and cosmetic components known to one skilled in the art are also contemplated in the composition of the present invention.

STD Treatments

The four most common STDs are syphilis, gonorrhea, chlamydia and trichomoniasis. A number of other causative agents are considered to cause STDs, such as the retroviruses HIV-1 and HIV-2; herpes simplex viruses; human cytomegalovirus; varicella-zoster virus; Epstein-Barr virus; and a variety of herpesvirus strains. In a further embodiment of the present invention, compositions are provided which include a preparation of free iron and an sexually-transmitted disease (STD) treatment component. STD treatment components for use in the compositions of the present invention include, but are not limited to various antibiotics (e.g., azithromycin, cefixime, ceftriaxone, ciprofloxacin, clindamycin, doxycycline, erythromycin, fluconazole, imiquimod, metronidazole, ofloxacin, podofilox); sulfated polysaccharides (e.g., carrageenans); zivoduine (AZT); cysteamine (2-aminoethanediol), cystamine, and derivatives thereof (U.S. Pat. No. 5646189 to Thoene); naphthalene sulfonate polymers; defensins, protegrins and cysteine-rich antimicrobial peptides (such as those disclosed in U.S. Pat. Nos. 4,705,777; 4,659,692; 4,543,252,and 6,159,936 to Lehrer et al.); branched polymethylether hydroquinone sulfonates and derivatives thereof; acetate phthalate or hydroxypropyl methylcellulose phthalate (see, for example, 6,165,493 to Neurath, et al.); β-lactoglobulin derivatives; tachyplesins (Nakamura, T. et al. J Biol. Chem. (1988) 263:16709-16713) and various lectins (see, for example, U.S. Pat. No. 6,159,174 to Oldham et al.).

These STD treatment compounds can be provided individually or as a combination thereof, either alone or in combination with a pharmaceutically acceptable carrier or diluent. Pharmaceutically acceptable compounds for use in this embodiment include, but are not limited to, those compounds listed previously for use in a lotion embodiment of the present invention.

Compositions for Treating Mucosal surfaces

The compositions of the present invention include compositions which can be used to treat a mucosal surface, such as an oral cavity, a vaginal cavity, a rectal surface, an intestinal surface, and the like. In yet another embodiment of the present invention, the composition of the present invention includes a preparation of free iron and an oral rinse. Oral rinse components include, but are not limited to, one or more of water, alcohol, benzoic acid, cetylpyridinium chloride, eucalyptol, glycerin, menthol, methyl salicylate, saccharin, sodium gluconate, polysorbate 80, thymol, xanthan gum, flavoring ingredients, and various dyes and/or colorants.

Carbohydrate-containing Compositions

In a further embodiment of the present invention, the composition of the present invention includes a preparation of free iron and a polysaccharide polymer. Optionally, the carbohydrate polymer is a polyamine, and preferably chitin, chitosan, or a derivative thereof. Alternatively, the carbohydrate polymer is a polymannan, such as β(1→4) acetylmannan or acemannan. However, other carbohydrates are also considered for use in the compositions of the present invention.

Various salts or organic complexes of the reduced and/or oxidized iron (or other transition metals) can be employed in the compositions of the present invention, including, but not limited to, acetate, ammonium citrate, ammonium oxalate, ammonium sulfate, bromide, chloride, citrate, fluoride, fumarate, hydroxide, iodide, nitrate, oxide, phosphate, pyrophosphate, sulfate, and tartrate. One preferred complex for use in the composition is iron citrate. For example, a composition of about 100 mM iron citrate and between about 0.01% chitosan and about 15% chitosan, at a pH of between 1-6 (optionally between pH 2 and pH 5), can be used to reduced microbial populations on a surface. Optionally, the composition comprises between about 0.01% and about 10% chitosan, preferably about 1% chitosan. Furthermore, the iron (or other transition metal) concentrations employed in these compositions can range from as low as about 0.1 nM to as high as about 1 M iron, as described previously in the methods of the present invention.

Optionally, the composition further includes a carrier material. One preferred carrier material is an oil, such as a vegetable oil (e.g., clove, oregano, coconut, olive, eucalyptus, tea tree (melaleuca) oils) or a mineral oil. The oil, for example, assists in solvating the polysaccharide polymer, and can optionally act as a further non-oxidative stress inducer. Other carrier materials are also contemplated, including various polymers and solvents.

Optionally, the composition includes a chelator. Exemplary chelators include, but are not limited to, chemical chelating moieties (such as EDTA) and biochemical structures (e.g., ferritin, transferrin, lactoferrin).

High Throughput Methodologies

The extent of synergistic reduction in microbial population using one or more transition metals and one or more stress inducers can be theoretically estimated; however, since different microbes have differing sensitivities to different stress inducers, and differing abilities to uptake transition metals, a more accurate estimate is determined by performing the methods of the present invention in a high throughput format. The present invention also provides methods which make possible the testing of a large number of variables, including individual and combinations of transition metals, individual stress inducers, combinations of stress inducers, durations of exposure, and concentrations of preparations. Additional parameters can be analyzed for effectiveness, such as relative sensitivities of different species, redox state of the transition metal, salt or organic complex employed, ionic strength of the solution, pH, temperature, characteristics of the surface being treated, presence of cofactors, biocide enhancers, or adjuvants, and the like, as desired by one of skill in the art. The optimum effectiveness of the compositions and methods disclosed herein can be determined in this manner. A further aspect of the present invention is a high throughput method for rapidly comparing the effectiveness of a plurality of microbiocidal compositions and parameters.

Kits

In an additional aspect, the present invention provides kits embodying the methods and compositions for reducing a microbial population on a surface, as described herein. The kits optionally comprise one or more of a) containers for packaging one or more composition elements, b) sponges, cloths, trays, pumps, spraying devices or other devices for contacting a surface with the compositions of the present invention, c) aqueous solutions for use with the composition, d) packaging materials, and the like. Furthermore, instructions, such as written directions or videotaped demonstrations detailing the use of the kits of the present invention, i.e., according to the methods set forth herein, are optionally provided with the kit.

In a further aspect, the present invention provides for the use of any composition or kit herein, for the practice of any method or assay herein, and/or for the use of any apparatus or kit to practice any assay or method herein.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method for reducing a microbial population on a surface, the method comprising:
providing a low concentration of free iron;
providing a non-oxidant stress inducer; and
bringing the surface into contact with the low concentration of free iron and the non-oxidant stress inducer, thereby reducing the microbial population on the surface.

2. The method of claim 1, wherein the low concentration of free iron comprises ferrous or ferric iron.

3. The method of claim 1, wherein the low concentration of free iron comprises between about 0.1 μM and about 100 mM free iron.

4. The method of claim 3, wherein the low concentration of free iron comprises between about 0.1 μM and about 1 mM free iron.

5. The method of claim 3, wherein the low concentration of free iron comprises about 1μM free iron.

6. The method of claim 1, wherein bringing the surface into contact with the low concentration of free iron and the non-oxidant stress inducer comprises coadministering the low concentration of free iron and the non-oxidant stress inducer.

7. The method of claim 1, wherein bringing the surface into contact with the low concentration of free iron and the non-oxidant stress inducer comprises removing 50% or more of the low concentration of free iron prior to bringing the surface into contact with the non-oxidant stress inducer.

8. The method of claim 7, wherein the bringing the surface into contact with the low concentration of free iron and the non-oxidant stress inducer comprises removing 75% or more of the low concentration of free iron prior to bringing the surface into contact with the non-oxidant stress inducer.

9. The method of claim 8, wherein bringing the surface into contact with the low concentration of free iron and the non-oxidant stress inducer comprises removing 95% or more of the low concentration of free iron prior to bringing the surface into contact with the non-oxidant stress inducer.

10. The method of claim 1, wherein the non-oxidizing stress inducer comprises one or more enzymes.

11. The method of claim 10, wherein the one or more enzymes comprise lysozyme, cellulase, endoxylanase, invertase, lactamase, pectinase, or zymolase.

12. The method of claim 1, wherein the non-oxidizing stress inducer comprises chitin or chitosan.

13. The method of claim 1, wherein the non-oxidizing stress inducer comprises polymannan, β(1→4) acetylmannan, acemannan, or aloe.

14. The method of claim 1, wherein the non-oxidizing stress inducer comprises one or more hypo-osmotic solutions.

15. The method of claim 1, wherein the non-oxidizing stress inducer comprises one or more hyper-osmotic solutions.

16. The method of claim 1, further comprises providing one or more biocide enhancers selected from the group consisting of riboflavin, flavenoids, photo-activatable compounds, phenols, cetyl pyridinium chloride, trisodium phosphate, hydrogen peroxide, bleach, one or more fatty acids, one or more organic acids, citric acid, and ascorbic acid.

17. The method of claim 1, wherein the surface comprises a food surface.

18. The method of claim 17, wherein the food surface comprises nuts, fruits or vegetables.

19. The method of claim 1, wherein the surface comprises an animal surface.

20. The method of claim 19, wherein the animal surface comprises an outer body surface, a digestive tract, or a combination thereof.

21. The method of claim 1, wherein the surface comprises a wound, an abrasion, a burn, or a damaged region of tissue.

22. The method of claim 1, wherein the surface comprises an environmental surface.

23. The method of claim 1, wherein the surface comprises a piece of medical equipment.

24. The method of claim 1, wherein the method further comprises: providing a siderophore.

25. The method of claim 24, wherein the siderophore comprises one or more of citrate, aerobactin, alcaligin, cepabactin, desferriferrichrysin, desferriferricrocin, desferriferrioxamine B, desferriferrioxamine E, coprogen, corrugatin, enterobactin, enterochelin, exochelin, ferrichrome, ferrioxamine, gallichrome, mycobaction, myxochelin, nocardamine, pseudobactin M114, pyoverdine, pyochelin, pseudobactin St3, rhizoferrin, rhodotorulic acid, schizokinen, pseudobactin 7NSK2, trencam, WCS, or vibriobactin.

26. The method of claim 1, wherein bringing the surface into contact comprises exposing the surface for between 0.05 hour and 24 hours.

27. The method of claim 1, wherein bringing the surface into contact comprises exposing the surface for between 1 hour and 7 days.

28. The method of claim 1, wherein the microbial population comprises a prokaryote, a fungus, a yeast, or a combination thereof.

29. The method of claim 28, wherein the microbial population comprises one or more of Bacillus, Burkholderia, Campylobacter, Chlamydia, Clostridium, Corynebacterium, Escherichia, Hemophilus, Helicobacter, Legionella, Listeria, Meningococcus, Mycobacterium, Mycoplasma, Neisseria, Pseudomonas, Salmonella, Shigella, Staphylococcus, Streptococcus, Trypanosoma, Vibrio, or Yersinia.

30. A method for reducing a microbial population on a food surface or a living tissue, the method comprising:
providing a composition comprising free iron;
providing a non-oxidant stress inducer; and
bringing the food surface or the living tissue into contact with the free iron and the non-oxidant stress inducer, thereby reducing the microbial population on the food surface or living tissue.

31. The method of claim 30, wherein bringing the food surface or the living tissue into contact with the free iron and the non-oxidant stress inducer comprises coadministering the free iron and the non-oxidant stress inducer.

32. The method of claim 30, wherein bringing the food surface or the living tissue into contact with the free iron and the non-oxidant stress inducer comprises removing 50% or more of the free iron prior to bringing the food surface or the living tissue into contact with the non-oxidant stress inducer.

33. The method of claim 32, wherein bringing the food surface or the living tissue into contact with the free iron and the non-oxidant stress inducer comprises removing 75% or more of the free iron prior to bringing the food surface or the living tissue into contact with the non-oxidant stress inducer.

34. The method of claim 33, wherein bringing the food surface or the living tissue into contact with the free iron and the non-oxidant stress inducer comprises removing 95% or more of the free iron prior to bringing the food surface or the living tissue into contact with the non-oxidant stress inducer.

35. The method of claim 30, wherein the free iron comprises between about 0.1 nM and about 1 M free iron.

36. The method of claim 35, wherein the free iron comprises between about 1 μM and about 100 mM free iron.

37. The method of claim 29, wherein the non-oxidizing stress inducer comprises chitin or chitosan.

38. The method of claim 30, wherein the non-oxidizing stress inducer comprises one or more enzymes.

39. The method of claim 38, wherein the one or more enzymes comprise lysozyme.

40. The method of claim 30, wherein providing the non-oxidizing stress inducer comprises exposing the food surface or living tissue to heat, irradiation, or osmotic shock.

41. The method of claim 30, further comprising providing one or more acids, bases, disinfectants, halides, organic solvents, oxidants, enzymes, antimicrobial agents, antibiotics, antiseptics and denaturants.

42. The method of claim 30, further comprising providing one or more biocide enhancers selected from the group consisting of riboflavin, flavenoids, photo-activatable compounds, cetyl pyridinium chloride, trisodium phosphate, hydrogen peroxide, bleach, one or more fatty acids, organic acids, citric acid, and ascorbic acid.

43. The method of claim 30, further comprises providing a siderophore.

44. The method of claim 43, wherein the siderophore comprises one or more of citrate, aerobactin, alcaligin, cepabactin, desferriferrichrysin, desferriferricrocin, desferriferrioxamine B, desferriferrioxamine E, coprogen, corrugatin, enterobactin, enterochelin, exochelin, ferrichrome, ferrioxamine, gallichrome, mycobaction, myxochelin, nocardamine, pseudobactin M114, pyoverdine, pyochelin, pseudobactin St3, rhizoferrin, rhodotorulic acid, schizokinen, pseudobactin 7NSK2, trencam, WCS, or vibriobactin.

45. The method of claim 30, wherein the surface comprises a food surface, and wherein bringing the surface into contact comprises exposing the surface for between about 30 seconds and about 5 minutes.

46. The method of claim 30, wherein the surface comprises a living tissue, and wherein bringing the surface into contact comprises exposing the surface for between about 4 hours and about 7 days.

47. The method of claim 30, wherein the microbial population comprises one or more of Bacillus, Burkholderia, Campylobacter, Chlamydia, Clostridium, Corynebacterium, Escherichia, Hemophilus, Helicobacter, Legionella, Listeria, Meningococcus, Mycobacterium, Mycoplasma, Neisseria, Pseudomonas, Salmonella, Shigella, Staphylococcus, Streptococcus, Trypanosoma, Vibrio, or Yersinia.

48. A method for reducing a microbial population on a surface, the method comprising:
providing a preparation comprising free iron;
providing one or more siderophores; and
bringing the surface into contact with the free iron and the one or more siderophores, thereby reducing the microbial population on the surface.

49. The method of claim 48, wherein the free iron comprises between about 0.1 nM and about 1 M free iron.

50. The method of claim 48, wherein the free iron comprises about 0.1 μm and about 100 mM free iron.

51. The method of claim 48, wherein the one or more siderophores comprises one or more of citrate, aerobactin, alcaligin, cepabactin, desferriferrichrysin, desferriferricrocin, desferriferrioxamine B, desferriferrioxamine E, coprogen, corrugatin, enterobactin, enterochelin, exochelin, ferrichrome, ferrioxamine, gallichrome, mycobaction, myxochelin, nocardamine, pseudobactin M114, pyoverdine, pyochelin, pseudobactin St3, rhizoferrin, rhodotorulic acid, schizokinen, pseudobactin 7NSK2, trencam, WCS, or vibriobactin.

52. The method of claim 48, wherein the surface comprises one or more of a food surface, an animal surface, an environmental surface, a piece of medical equipment, a wound, an abrasion, a bum, or a damaged region of tissue.

53. The method of claim 52, wherein the animal surface comprises an outer body surface, a digestive tract, or a combination thereof.

54. The method of claim 48, further comprising providing a non-oxidant stress inducer.

55. The method of claim 54, wherein the non-oxidizing stress inducer comprises chitin or chitosan.

56. A composition for reducing a microbial population on a surface, the composition comprising a preparation of free iron and a non-oxidant stress inducer, wherein the non-oxidant stress inducer comprises chitin, chitosan, a chitin derivative, or combinations thereof.

57. The composition of claim 56, further comprising an oil.

58. The composition of claim 56, further comprising EDTA.

59. A composition for reducing a microbial population on a surface, the composition comprising a preparation of free iron and one or more of a wound dressing component, a lubricant, an STD treatment component, or an oral rinse.

60. The composition of claim 59, further comprising a non-oxidant stress inducer.

61. The composition of claim 59, further comprising an iron-binding compound.

62. The composition of claim 61, wherein the iron-binding compound comprises a chelator.

63. The composition of claim 62, wherein the chelator comprises a siderophore.
